(19) United States
(12) Patent Application Publication
(10) Pub. No.: US 2021/0299181 A1
(43) Pub. Date: Sep. 30, 2021

(54) ANUCLEATED CELLS FOR THE TREATMENT OF DISEASES

(71) Applicant: Platelet Biogenesis, Inc., Watertown, MA (US)

(72) Inventors: Sunita R. Hett, Watertown, MA (US); Karen Weisinger, Watertown, MA (US); Yang Peng, Watertown, MA (US); Kyle P. McLaughlin, Watertown, MA (US); Christian G. Peters, Watertown, MA (US); Andrew R. Zukauskas, Watertown, MA (US); Brenden W. Smith, Watertown, MA (US); Silvia Giannini, Watertown, MA (US); Po-Shun Lee, Watertown, MA (US)

(21) Appl. No.: 17/213,796

(22) Filed: Mar. 26, 2021

Related U.S. Application Data
(60) Provisional application No. 63/000,866, filed on Mar. 27, 2020.
(60) Provisional application No. 63/104,871, filed on Oct. 23, 2020.
(60) Provisional application No. 63/125,490, filed on Dec. 15, 2020.
(60) Provisional application No. 63/142,586, filed on Jan. 28, 2021.
(60) Provisional application No. 63/024,649, filed on May 14, 2020.

(51) Int. Cl. A61K 35/19; G01N 33/564; C12N 5/078; A61P 37/06

(52) U.S. Cl. CPC A61K 35/19; G01N 33/564; G01N 2800/24; A61P 37/06; C12N 5/0644

(57) ABSTRACT

The present disclosure takes advantage of non-naturally existing, novel, anucleated platelets or platelet-like cells or platelet variants (collectively referred to as PLCs) or derivatives thereof (i.e., genetically engineered), which share at least one common receptor, ligand or an antigen with endogenous cells that are targets for autoantibodies and effectuate their clearance. The present disclosure also takes advantage of viral adhesion or entry receptors or genetically engineered PLCs or derivatives thereof, which are engineered to express viral adhesion or entry receptors, that specifically binds to viral proteins on viruses or viral particles and effectuate their clearance.

### RELATED APPLICATIONS

This application claims priority to U.S. Provisional Patent Application Ser. No. 63/000,866, filed Mar. 27, 2020; U.S. Provisional Patent Application Ser. No. 63/104,871, filed Oct. 23, 2020; U.S. Provisional Patent Application Ser. No. 63/125,490, filed Dec. 15, 2020; U.S. Provisional Patent Application Ser. No. 63/142,586, filed Jan. 28, 2021; and U.S. Provisional Patent Application Ser. No. 63/024,649, filed May 14, 2020, the entire contents of each of which are herein incorporated by reference.

### FIELD

The present disclosure relates to non-naturally existing, novel, anucleated cells or derivatives thereof for treating diseases. More specifically, the present disclosure is directed to non-naturally existing novel anucleated platelets or platelet-like cells or platelet variants or derivatives thereof or extracellular vesicles derived therefrom or derivatives thereof, displaying receptors, ligands or antigens, or acting as vehicles, to target proteins, autoantibodies, antigens, viral or bacterial proteins, or other disease molecules such as biological or chemical toxins, and their role in treating or ameliorating diseases, such as autoimmune diseases, viral diseases, bacterial diseases or other diseases.

### BACKGROUND

Autoimmune diseases are debilitating diseases caused mostly by unknown triggers that lead to undesired immune system activity that instead of fighting infections attack the body's own tissues ultimately leading to tissue destruction. Inappropriate humoral and cellular immune responses mediate the tissue damage in autoimmune diseases. One part of the humoral arm of the immune assault is caused by autoantibodies. Finding a cure for autoimmune diseases is a challenge and has met with little or very limited success. Clearly, there is a need to find new treatments that will prevent the immune cells from attacking the very cells (e.g., platelets) that they are meant to protect thereby alleviating the sufferings of patients suffering from autoimmune diseases. Likewise, there is an urgent need to develop therapies against continuously evolving viral or other diseases (e.g., bacterial or toxin-related diseases). While the role of platelets are being understood in fighting against pathogens, including viruses, there are several encumbrances associated with their use. For example, platelet loss during autoimmune, viral of or other diseases requires continuous need for human donors to replace the lost platelets, which often results in shortages in supply. Short life-span of platelets, frequent contamination, inconsistencies in quality are among other disadvantageous factors associated with platelets. Herein are described novel ways to treat or ameliorate autoimmune, viral or other diseases with novel, non-naturally existing, anucleated platelets or platelet-like cells or platelet variants or the genetically engineered derivatives thereof. As well as described herein are novel ways to treat or ameliorate autoimmune, viral or other diseases with the extracellular vesicles or genetically engineered derivatives thereof either alone or in combination with the novel, non-naturally existing, anucleated platelets or platelet-like cells or platelet variants or the genetically engineered derivatives thereof.

### SUMMARY

Described herein, in some embodiments of the invention, are methods for treating or ameliorating autoimmune diseases with non-naturally existing, novel, anucleated platelets or platelet-like cells or platelet variants (collectively referred to as “PLCs” or in its singular form: “PLC”) or derivatives thereof (e.g., genetically engineered PLCs) that share at least one common receptor, ligand or an antigen with an endogenous autoantigenic cell population with, for example but not limited to, bone marrow derived platelets, nuclear and cell membrane phospholipid components, such as chromatin or ribonucleoprotein particles, insulin-producing β cells, or autoantigenic cell population bearing autoantigenic peptides, such as but not limited to, GPIIb/IIIa, α-subunit of Acetylcholine Receptor (AChR), Aquaporin-4, ADAM metallopeptidase with thrombospondin type 1 motif 13 (ADAMTS13), Anti-N-methyl-D-aspartate receptor (anti-NMDAR), Phospholipase A2R that may be presented at different levels on different MHC molecules or otherwise and are targets for autoantibodies. Advantageously, the at least one common receptor, ligand or an antigen, whether endogenously or exogenously expressed on the PLCs or derivatives thereof, specifically bind to autoantibodies that otherwise target the endogenous autoantigenic cell population. Binding of the autoantibodies to the PLCs or derivatives thereof substantially frees the endogenous cell population (e.g., endogenous platelets) from damage caused by the autoantibodies. Upon elimination or decrease in the autoantibody population in a patient induced by the PLCs or derivatives thereof (e.g., coupling of PLCs with autoantibodies to form PLC-autoantibody complex), the endogenous cell population can perform their physiological role thereby eliminating or ameliorating autoimmune diseases. For example, PLCs or derivative thereof can bind to autoantibodies against donor platelets thereby freeing the donor platelets to perform their physiological roles.

Several advantages are provided by the PLCs or derivatives thereof of the present disclosure. The PLCs or derivatives thereof are artificially generated cells that endogenously express many receptors, ligands or antigens or such receptors, ligands or antigens can be genetically engineered into the PLCs to share common autoantigenic, viral, bacterial or other receptors, ligands, or antigens found on endogenous cell populations (e.g., autoantigenic cell populations, viral cell population) that trigger autoimmune responses or viral-induced disease. The PLCs or derivatives thereof are capable of systemic distribution (i.e., can be distributed into interstitial and intracellular fluids) for targeting undesired autoantibodies or viral proteins, for example to bind to such undesired autoantibodies or viral proteins (e.g., forming a PLC-autoantibody complex or PLC-viral protein complex) for their removal or degradation. The PLCs may also utilize their migratory role i.e., travelling from a first location, where the PLCs are administered, to a second location i.e., a diseased location where the undesired autoantibodies or viral proteins are present to bind to such undesired autoantibodies or viral proteins (e.g., forming a PLC-autoantibody complex or PLC-viral protein complex) such that an autoimmune (AI) disease or disease inducing viruses can be targeted with ease. Another advantage of the PLCs or derivatives thereof is that is that they can travel through blood flow potentially without immunogenicity. Furthermore, the PLCs or derivatives thereof, because of their large surface area, advantageously provide greater receptor, ligand or an antigen concentration or may carry additional therapeutic agents, such as an antibody or a fragment thereof, to treat the AI diseases or viral diseases. Yet, another advantage is that PLCs or derivatives thereof are essentially allogeneic, are not cancerous, or do not exhibit uncontrolled growth or tumor formation in-vivo, thereby facilitating PLC-based therapies. Moreover, PLCs are artificially generated and can be advantageously made in unlimited supply, consistent in quality and efficacy.

Thus, in some embodiments, PLCs or derivatives thereof bind to and clear proteins or toxins (e.g., autoantibodies or viral particles or bacterial proteins, or biological or chemical toxins) in circulation. For example, the PLCs or derivatives thereof essentially migrate to the liver as compared to donor platelets which migrate to spleen. In the liver, the PLCs or derivatives thereof induce clearance of the diseased molecules, such as but not limited to, toxins (biological or chemical) autoantibodies, proteins, peptides or antigens. In some embodiments, PLCs or derivatives thereof show rapid kinetics of clearance thereby facilitating clearance of toxic molecules (e.g., autoantibodies, proteins, peptides or antigens) from circulation. In some embodiments, PLCs or derivatives thereof play a role in liver-mediated antigen tolerization. In some embodiments, the use of the PLCs or derivatives thereof avoid global depletion of normal proteins as only pathogenic proteins (e.g., autoantibodies or viral proteins) are neutralized. In some embodiments, PLC-toxins can be generated from the PLCs or derivatives thereof. PLC-toxins are made from a toxin attaching (for example, by genetic engineering or by bioconjugation or chemical conjugation) to a PLC or a PLC derivative target proteins present on a target cell (e.g., cancer cells, autoimmune antibodies or cells generating such antibodies, or viruses or bacteria or particles or proteins thereof). Here the PLC portion of the molecule directs it to a specific antigenic determinant on a target cell; the molecule is then internalized, or is complexed and a cytotoxic reaction occurs.

Platelet autoantibodies are detected in approximately 50% of patients and generally target the fibrinogen receptor αIIbβ3 or the receptor for von Willebrand factor (VWF), the glycoprotein (GP) Ib-V-IX complex. Anti-αIIbβ3 antibodies (70-80% of cases) are thought to induce thrombocytopenia through increased platelet clearance by Fcγ receptor-bearing macrophages. Autoantibodies against GPIb-V-IX (20-40% of cases) often induce a more severe fall in platelet count. PLCs or derivatives thereof expressing such receptors can advantageously disrupt such deleterious interactions between endogenous cells and autoantibodies. For example, the PLCs share one or more common cell surface receptors, such as but not limited to, CD41 and/or CD-61 i.e., PLC^{CD41} and/or PLC^{CD41-CD61}, with bone marrow derived endogenous platelets. In bone marrow derived endogenous platelets CD41 and CD61 are targets for glycoprotein IIb/IIIa autoantibodies. In some embodiments, PLCs share CD42b and/or CD42a receptors (i.e., PLC^{CD42b} or PLC^{CD42} or PLC^{CD42b-Cd42a}) with bone derived platelets, which are targets for glycoprotein Ib/IX autoantibodies. Binding of the autoantibodies to endogenous bone marrow derived platelets depletes their physiological efficacy, a leading cause for various autoimmune diseases, for example, immune thrombocytopenia (ITP). Here the PLCs or derivatives thereof, which share the CD41 and/or CD-61 and or CD42a autoantigenic receptors with bone marrow derived platelets, specifically binds to the glycoprotein IIb/IIIa autoantibodies or PLCs or derivatives thereof, which share CD42a autoantigenic receptors with bone marrow derived platelets, specifically binds to the glycoprotein Ib/IX autoantibodies to form, for example, PLC^{CD41-CD61}-glycoprotein IIb/IIIa autoantibody complex or to form a PLC^{CD42b-CD42a}-glycoprotein Ib/IX autoantibody complex. In doing so, PLCs or derivatives thereof free the endogenous bone marrow derived platelets from the attack by the autoantibodies to free the donor platelets to play their physiological roles thereby eliminating of ameliorating the threat of inducing an autoimmune disease or disorder, such as but not limited to, immune thrombocytopenia (ITP), Myasthenia Gravis, Acquired Thrombotic Thrombocytopenic Purpura (aTTP), Membranous Nephropathy, Neuromyelitis Optica Spectrum Disorder, N-methyl D-aspartate (NMDA) receptor (NMDAR) Encephalitis, among others. Moreover, the PLCs or derivatives, while unique in their characteristics and functionalities retain many of functional indices of bone marrow derived platelets (e.g., some of the growth factors, receptors, ligands or antigens), which makes the PLCs or derivatives thereof uniquely placed to substitute for the donor platelets. Hence, the PLCs or derivatives thereof, besides suppressing the autoantibodies, may complement the physiological roles of an endogenous cell (e.g., bone marrow derived platelets) that are targeted by autoantibodies in autoimmune diseases.

In some embodiments, PLCs or derivatives thereof provide a ligand that mimics a ligand on an endogenous cell that is a target for an autoantibody or a viral protein. In some embodiments, the autoantibody is attracted to the PLCs' ligand (i.e., autoantibody couples to the PLCs ligand), binding thereto (i.e., PLC-ligand-autoantibody complex) thereby freeing the endogenous cells to perform their physiological roles, thereby preventing or ameliorating autoimmune diseases.

In some embodiments, PLCs or derivatives thereof provide a receptor that mimics a receptor on an endogenous cell that is a target for an autoantibody or a viral protein. In some embodiments, the autoantibody is attracted to the PLCs' receptor (i.e., autoantibody couples to the PLCs receptor), binding thereto (i.e., PLC-receptor-autoantibody complex) thereby freeing the endogenous cells to perform their physiological roles, thereby preventing or ameliorating autoimmune diseases.

In some embodiments, PLCs or derivatives thereof provide an antigen that mimics an antigen on an endogenous cell that is a target for an autoantibody or a viral protein. In some embodiments, the autoantibody is attracted to the PLCs' antigen (i.e., autoantibody couples to the PLCs antigen), binding thereto (i.e., PLC-antigen-autoantibody complex) thereby freeing the endogenous cells to perform their physiological roles, thereby preventing or ameliorating autoimmune diseases.

In some embodiments of the present disclosure, the PLCs or derivatives thereof have a higher receptor concentration than the concentration of the same autoantigenic endogenous receptors on endogenous cells. For example, PLCs have a higher concentration of CD63 as compared endogenous platelets thereby attracting greater concentration of autoantibodies to bind to the PLCs and freeing the endogenous cells to perform their physiological roles.

In some embodiments, PLCs may be produced by genetically engineered PLC-producing progenitor cells such that PLCs express a higher number of certain receptors, ligands or antigens (e.g., CD41/CD61 and CD42a), than the comparable number of receptors, ligands or antigens that the PLCs already express as compared to autoantigenic endogenous cells. Such PLCs provide the advantage of more effectively targeting autoantibodies by providing excess receptors, ligands or antigens to which the autoantibodies can bind to. For example, PLCs overexpressing CD41/CD61 or/CD42a will attract greater number of autoantibodies that are directed against platelet membrane glycoprotein (GP) complexes, thereby freeing endogenous cells from harmful effects of the autoantibodies, essential to the treatment of autoimmune diseases.

In some embodiments, the PLCs or derivatives thereof have a lower receptor concentration than the concentration of the same autoantigenic endogenous receptors, which advantageously can be used to deliver therapeutic agents that can complement the PLCs or derivatives thereof in treating autoimmune diseases. In some embodiments, concentrations of such receptors can be enhanced in a manner discussed herein.

Irrespective of the concentration of receptors, ligands or antigens on the PLCs or derivatives thereof that is commonly shared with the autoantigenic endogenous cells, the PLCs or derivatives thereof may be used in combination with other therapeutic agents that are used in treating or ameliorating autoimmune diseases.

In some embodiments, the present disclosure takes advantage of existing viral adhesion or entry receptors, ligands or antigens on the PLCs or derivatives thereof (e.g., complement receptor type 2 (CR2) on the PLC cell surface that facilitate a direct interaction of PLCs with viral pathogens (e.g., Epstein-Barr virus (EBV)), or genetically engineered PLCs, which are engineered to express viral adhesion or entry receptors (e.g., ACE-2) that specifically bind to viral proteins, for example, structural spike (S) protein or to the receptor-binding domain (RBD) in SARS-CoV-2 S protein, or bind to viral nonstructural proteins.

Viral adhesion or entry receptors expressed naturally on PLCs, or genetically engineered into the PLCs, can bind to viral proteins on viral pathogens. Viral pathogens include but are not limited to corona virus, influenza virus, reovirus, MERS corona virus (MERS-CoV), Polyomavirus, HIV, measles virus, reovirus, rhinovirus, adenovirus, poliovirus, coxsackievirus B (CVB), rotavirus, adenovirus, West Nile virus (WNV), human metapneuomovirus (hMPV), foot-and-mouth disease virus (FMDV), herpes simplex virus (HSV), human cytomegalovirus HCMV or human herpesvirus-8.

PLCs or derivatives thereof also provide the advantage of delivering to target pathogenic viruses more viral adhesion or entry receptors because of their large surface area as compared to other therapeutic molecules such as compared to bone marrow derive platelets. Other advantages include rapid and reliable testing for viruses or viral particles by the PLCs or the genetically engineered PLCs, which could help to preserve health in a quarantined patient population and to protect a healthy population from being infected by the patient population.

In some embodiments of the invention, the genetically engineered PLCs (i.e., PLC derivatives) take advantage of several receptors (exogenously or endogenous), such as but not limited to, PLCs' toll like receptors (TLR 1, TLR 2, TLR 3, TLR 4, TLR 6, TLR 7, TLR 8 or TLR 9) that detect and bind viral components at the cell surface and viral nucleic acids. In some embodiments, PLC degranulation is triggered by the viral interaction, which induces rapid direct interaction of PLCs and neutrophils. Here it is expected that the PLCs will decrease viral counts independently of or in a TLR-dependent fashion.

In some embodiments, the genetically engineered PLCs or the PLCs or derivatives thereof take advantage of complement receptors (genetically expressed or endogenous to PLCs), such as but not limited to, CR2, CR3, CR4, C3aR, C5aR, gC1qR and cC1qR. These proteins act as receptor for viral pathogens and implement multiple direct and indirect functions in antipathogen host defense, including, cell lysis, opsonization and chemotaxis.

In some embodiments, PLCs or derivatives thereof take advantage of PLC-activation after pathogen particles bind to the PLCs and the PLCs phagocyte infectious microorganisms (e.g., virus). The activated PLCs undergo degranulation and release of a variety of inflammatory mediators, chemokines and cytokines, such as but not limited to, adenosine diphosphate (ADP), thromboxane A2 (TXA2), serotonin, cytokines (IL-1β, TNFα, TGF-β, CD40L) and chemokines (CXCL1 (GROa), CXCL4/PF4, CXCL5, CXCL7, CXCL12, CXCL14, CCL5 (RANTES), CCL3 (MIP1a), MCP-3 (CCL7)) from their granules. These PLC-derived chemokines and cytokines stimulate migration of monocytes and enhance phagocytosis of pathogens, such as viruses and bacteria, such as but not limited to, viruses belonging to the Coronaviridae family.

In some embodiments, PLCs or derivatives thereof take advantage of the host defense mechanisms in response to viral infections, which lead to PLC activation. PLCs or derivatives thereof are rich in α-granules, which provide PLC-mediated host defense mechanisms, as they contain kinocidins and microbicidal proteins important for antiviral host defense. Kinocidins have immune-modulatory functions including chemotaxis and activation of various immune cells. They can differentiate between host and pathogen cell membranes and distinct pathogen spectra under different pH conditions. The host defense mechanisms, in response to viral infections, also take advantage of the chemokines released during viral infections, such as but not limited to, CCL5 and CCL3. CCL5 and CCL3 have been identified as major viral suppressive factors produced by cytotoxic T-lymphocytes. It is contemplated that PLC-derived CCL5 and CCL3 could play the same role, i.e., suppress viral activity, e.g., COVID-19 activity.

Whether it is the clearance of autoantibodies or viral pathogens coupled to the PLCs or derivatives thereof, in some embodiments PLCs or derivatives thereof take the advantage of clearing the harmful autoantibodies or viral pathogens that is mediated by liver. An added advantage of liver-mediated clearance of PLC-coupled autoantibodies or viral pathogens is that it facilitates rapid clearance and degradation of the autoantibodies or viral pathogens rendering them harmless. For example, efficacy of the PLCs/EVs or derivatives thereof is targeting 90% response within 24 hours (for acute) and >90% response rate within 4 weeks, sustained >24 weeks (for chronic).

In some embodiments, the present disclosure provides non-natural extracellular vesicles (EVs) that are admixtured with the PLCs, during the making of the PLCs. Extracellular vesicles (EV) comprise microvesicles (MV) or exosomes or a combination thereof, are smaller in size as compared to PLCs, are made along with platelet like cells and are biologically active. Non-natural EVs comprise microvesicles (MV) or exosomes or a combination thereof. Each component in the admixture, i.e., PLCs, microvesicles and exosomes can substantially be isolated into individual components from the admixture. The extracellular vesicles (EV) function as a transport and delivery system for bioactive molecules, play a role in hemostasis and thrombosis, inflammation, malignancy infection transfer, angiogenesis, and immunity. Thus, in some embodiments, EVs may complement PLCs and their combination is an even richer resource for PLC-based therapeutic applications.

In some embodiments, the EVs of the present disclosure comprise exosomes, 30-200 nm in diameter carrying multifarious molecules such as proteins, lipids, and RNAs either on their surface or within their lumen. Exosomes play a role in stimulating tissue regeneration, in many in-vitro and in-vivo models, demonstrating that they can confer proangiogenic, proliferative, antiapoptotic and anti-inflammatory actions through transporting RNA and protein cargos. Thus, in some embodiments, exosomes make it even a richer resource for PLC-based therapeutic applications.

In some embodiments, the EVs of the present disclosure comprise microvesicles (MV), 200-1000 nm in diameter, carrying multifarious molecules such as proteins, lipids, and RNAs either on their surface or within their lumen. EV play a role in stimulating tissue regeneration, in many in-vitro and in-vivo models, demonstrating that they can confer proangiogenic, proliferative, antiapoptotic and anti-inflammatory actions through transporting RNA and protein cargos. Thus, in some embodiments, EVs make it even a richer resource for PLC-based therapeutic applications.

In some embodiments of the present disclosure, PLCs or the PLC-producing cells (e.g., iPSCs or Megakaryocytes) are genetically engineered to express a receptor, a ligand or an antigen, recognized by autoantibodies or viral proteins (such genetically engineered PLCs are referred to as PLC derivatives (i.e., derivatives thereof) throughout the application). For example, PLCs may be lacking a common receptor with an autoantigenic endogenous cell but upon being genetically engineered provide commonly shared receptors, ligands or antigens between engineered PLCs and autoantigenic endogenous cells or viral proteins to bind to autoantibodies or viral proteins that otherwise would bind to autoantigenic endogenous cells or to endogenous organs & tissues in the body. Here it is also contemplated that the PLCs or the PLC-producing cells (e.g., iPSCs or Megakaryocytes) may be genetically engineered to provide a higher concentration of receptors than that are already present therein. For example, PLCs or PLC producing progenitor cells can be genetically engineered to express higher concentration of CD41+ and/or CD-61+ i.e., PLC^{CD41+} and/or PLC^{CD41+-CD61+} than that are originally found on the PLCs to form PLC^{CD41+-CD61+}-glycoprotein IIb/IIIa autoantibody complex or form a PL^{CD42a+}-glycoprotein Ib/IX autoantibody complex. In some embodiments, PLCs or PLC producing progenitor cells can be genetically engineered to express ADAMTS13 (PLC^{ADAMTS13}, which forms PLC^{ADAMTS13}-autoantibody complex for the removal of autoantibodies responsible for the disease aTTP), NMDA receptor (PLC^{NMDA receptor}, which forms PLC^{NMDA receptor}-autoantibody complex for the removal of autoantibodies responsible for the disease NMDAR encephalitis), alpha subunit of AchR (PLC^{alpha subunit of AchR}, which forms PLC^{alpha subunit of AchR}-autoantibody complex for the removal of autoantibodies responsible for the disease Myasthenia gravis), aquaporin 4 (PLC^{aquaporin 4}, which forms PLC^{aquaporin 4}-autoantibody complex for the removal of autoantibodies responsible for the disease Neuromyelitis Optica Spectrum Disorder) or TSH-receptor (PLC^{TSH-receptor}, which forms PLC^{TSH-receptor}-autoantibody complex for the removal of autoantibodies responsible for the disease Pemphigus vulgaris), which are poorly expressed or are lacking in expression in the PLCs. Likewise, PLCs can be engineered to express receptors, ligands or antigens that are targets for bacteria or viruses.

In some embodiments, the present disclosure provides a method of treating a patient suffering from autoimmune diseases, the method comprising administering to the patient a therapeutically effective amount of PLCs or derivatives thereof that share with an endogenous cell, the same autoantigen bearing receptors, ligands or antigens, sufficient to treat a patient suffering from autoimmune (AI) diseases (e.g., ITP, Myasthenia Gravis, Acquired Thrombotic Thrombocytopenic Purpura (aTTP), Membranous Nephropathy, Neuromyelitis Optica Spectrum Disorder, N-methyl D-aspartate (NMDA) receptor (NMDAR) Encephalitis, Graves Opthalmology, among others).

In some embodiments of the present disclosure is provided a method of treating a patient suffering from an autoimmune disease, the method comprising administering to the patient a therapeutically effective amount of PLCs or derivatives thereof, that share with an endogenous cell the same autoantigen bearing receptors, ligands or antigens, in combination with one or more agents used in the treatment of autoimmune (AI) diseases, sufficient to treat a patient suffering from the AI diseases.

The present disclosure also provides a composition (e.g., a pharmaceutical composition) comprising novel PLCs or derivatives thereof and a carrier (a pharmaceutically acceptable carrier). The present disclosure additionally comprises a composition (e.g., a pharmaceutical composition) comprising novel PLCs and/or derivatives thereof, and a carrier (a pharmaceutically acceptable carrier), further comprising a second therapeutic agent. The compositions are useful for treating autoimmune, bacterial or viral diseases in a mammal (e.g., human), discussed below in greater detail.

In some embodiments, the PLCs or derivatives thereof may be co-administered with ex-vivo derived endogenous cells, that otherwise are endogenously autogenic. Here, the ex-vivo derived endogenous cells may be derived from a healthy donor. In some embodiments, prior to co-administering, the ex-vivo derived endogenous cells may be modified in a manner that complements the PLCs or derivatives thereof in diminishing the harmful role of autoantibodies in an autoimmune disease. For example, when the PLCs or derivatives thereof are co-administered with ex-vivo derived endogenous cells the PLCs or derivatives thereof not only bind to the autoantibodies (i.e., form PLC-autoantibody complex), but with the assistance of donor platelets assist in restoring the physiological role of the endogenous cell population affected by the autoantibodies.

In some embodiments, the present disclosure provides a method of treating a patient suffering from a pathogenic infection (e.g., viral infection), the method comprising administering to the patients in therapeutic amount of PLCs or derivatives thereof (e.g., the genetically engineered PLCs) of the present disclosure thereby causing amelioration of or treatment of the viral infection. In some embodiments, the method comprises administering a second therapeutic agent.

In some embodiments, the methods of the present disclosure are used to disrupt the life cycle of a pathogen. In some embodiments, the methods are used to disrupt the life cycle of a virus having an RNA genome, for example a retrovirus, by targeting the RNA genome directly. In some embodiments, a viral genome transcript is targeted, including transcripts of individual viral genes. The methods also can be used to down regulate a gene in a host cell, where the gene is involved in the viral life cycle, for example, a receptor or co-receptor necessary for viral entry into the host cell or enzymes that make up replicase complex or protein translation enzymes or Ribosomal proteins.

In some embodiments, the present disclosure provides a pharmaceutical composition comprising PLCs or derivatives thereof (e.g., the genetically engineered PLCs) and a pharmaceutically acceptable carrier. In some embodiments, the pharmaceutical composition further comprises a second therapeutic agent.

In some embodiments, the present disclosure provides kit comprising PLCs or derivatives thereof (e.g., the genetically engineered PLCs) of the present disclosure described herein. In some embodiments of the present disclosure, the PLCs are engineered to recognize one or more viral receptors or protein for an early diagnostic of viral infections where the PLCs or derivatized PLCs (e.g., genetically engineered PLCs) recognize such viral receptors or proteins.

Advantageously, the compositions for use according to the present disclosure are capable of treating, ameliorating or diagnosing a viral infection. Advantageously, this means that more patients can be treated for the viral infections (i.e., seropositive patients) and the spread of infection can be contained.

In some embodiments, the present disclosure provides a diagnostic method comprising: (a) obtaining a sample from an individual (e.g., a healthy individual, an asymptomatic individual or a patient); (b) admixing with the sample a composition comprising at least one anti-viral agent (for example PLCs or derivatives thereof that exogenously or endogenously express one or more viral entry receptor proteins); (c) determining the presence or absence of a virus or viral particles or viral peptides or viral nucleic acids or a combination thereof in the sample.

The present disclosure also provides a method of screening for a virus, said method comprising: (a) obtaining a sample from a patient; (b) admixing with said sample a composition comprising an anti-viral agent (for example PLCs or derivatives thereof that exogenously or endogenously express one or more viral entry receptor proteins); (c) determining the presence or absence of a virus or viral particles or viral peptides or viral nucleic acids or a combination thereof in the sample.

### BRIEF DESCRIPTION OF FIGURES

The presently disclosed embodiments will be further explained with reference to the attached drawings, in which:

FIGS. 1A-1C show fluorescently labeled antibodies PAB-1 and 96-2C1 (Anti-CD41/61 antibodies) (FIGS. 1A and 1B) can be quantified in mouse blood (FIG. 1A) and plasma (FIG. 1B), respectively. FIG. 1C shows PLCs (PB101) binds fluorescently labeled Anti-CD41/61 antibodies.

FIGS. 2A-2B show PLCs clear anti-CD41 antibody (96-2C1) in-vivo.

FIGS. 3A-3B further show that PLC-dependent anti-CD41 antibody (96-2C1) clearance occurs in the liver.

FIGS. 4A-4B show PLCs clear anti-CD41/61 antibody (PAB-1) in-vivo.

FIGS. 5A-5B show PLC-dependent anti-CD41/61 antibody (PAB-1) clearance occurs in the liver.

FIG. 6 shows PLCs bind anti-CD41/61 antibody (PAB-1) in-vivo.

FIGS. 7A-7C show PB101 is titratable and a reduction in EC50 is achieved with anti-CD41/61 antibody dosing comparable to levels in ITP patients.

FIGS. 8A-8B show PB101 uptake by the liver specifically results in TPO upregulation in-vivo.

FIG. 9 shows PLCs expressing high levels of glycoproteins (e.g., CD41, CD61) on the surface that can bind and clear the pathogenic autoantibodies from circulation and restore donor platelets and megakaryocytes to their physiologic functions.

FIG. 10 is a negative control where experiments were performed with K562 cells, a human leukemia cell line lacking glycoprotein IIb/IIIa receptors.

FIG. 11 shows that increasing numbers of PLCs leads to enhanced antibody reduction.

FIGS. 12A-12D illustrate that PLCs' mechanism of action lends itself to expansion of the platform to diverse antibody mediated autoimmune diseases. FIGS. 12A and 12B show the presence of desmoglein 2 and 3 receptors, respectively, endogenous to the PLCs. FIG. 12C summarizes some of the autoimmune disease receptors that are (1) endogenously expressed in the PLCs or (2) can be exogenously expressed in the PLCs. FIG. 12D is an exemplification, which shows concentration of some of the autoimmune disease receptors in the PLCs vs donor platelets.

FIGS. 13A-13B show PLCs express specific antigens that may bind and clear diverse disease-causing circulating antibodies. Specifically, FIG. 13A shows clearance of desmocollin antibody and FIG. 13B shows clearance of desmoglein antibody, which are indications for Pemphigus Vulgaris.

FIGS. 14A and 14B(i)-14B(v) outlines the constructs used for generating ePLCs for various AI indications.

FIGS. 15A and 15B show lentiviral packaging cells (HEK) express exogenously introduced acetylcholine receptor (AchR) epitopes, which could be utilized by the ePLCs to siphon Acetylcholine receptor (AChR) antibodies i.e., autoantibodies produced by the immune system that mistakenly target proteins called acetylcholine receptors that are located on muscles.

FIGS. 16A and 16B show FLAG tag detection in ‘AchR’ (extracellular domain of (Acetylcholine receptor) transfected HEK packaging cells.

FIGS. 17A-17B and 17C-17D show an example of eEVs/ePLCs expressing exogenous genes, exemplified by IL-12. In FIGS. 17A and 17B, genetically engineered eEVs express IL-12. In FIGS. 17C and 17D, genetically engineered PLCs (as well as engineered megakaryocyte like cells (MLCs), generated from genetically engineered iPSc) express IL-12.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

In the following, the elements of the present disclosure will be described. These elements are listed with specific embodiments; however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. The terms “a” and “an” and “the” and similar reference used in the context of describing the present disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). The terms used herein are also defined as described in “A multilingual glossary of biotechnological terms: (IUPAC Recommendations)”, H. G. W. Leuenberger, B. Nagel, and H. Kolbl, Eds., (1995) Helvetica Chimica Acta, CH-4010 Basel, Switzerland. As used herein, the following terms have the meanings ascribed to them below, unless specified otherwise.

The terms “agent,” “therapeutic agent,” “therapeutic composition,” “drug,” or “therapeutic” can be used interchangeably and are meant to include any small molecule chemical compound, antibody, nucleic acid molecule, or polypeptide, or fragments thereof.

“Non-natural” as used herein refers to manufactured, created, or constructed by human beings, artificial, or mimicking something that exists in nature.

“Derivatives”, as used herein, refer to genetically engineered PLCs (ePLCs) or genetically engineered extracellular vesicles (eEVs) or a combination thereof for therapeutic use, inclusive of PLC precursor cells (e.g., pluripotent stem cells genetically engineered in a manner such that that the PLCs or extracellular vesicles produced by these PLC/EV precursor cells produce a molecule of interest in the PLCs or extracellular vesicles or in both, or in any other modification of described herein. Derivatives are also inclusive of bioconjugates of PLCs and extracellular vesicles or bioconjugates of genetically engineered PLCs and extracellular vesicles. Derivatives are also inclusive of cargo carrying PLCs and extracellular vesicles or cargo carrying genetically engineered PLCs and extracellular vesicles. Here, for example, the PLCs or extracellular vesicles can be first subjected to genetic engineering, then their cargo carrying capacity is utilized. In other words, the term derivative is inclusive of any modification, genetic, chemical or a combination thereof otherwise of the PLCs, genetically engineered PLC, extracellular vesicles or genetically engineered extracellular vesicles.

As used herein “progenitor cells” refers to IPSC-derived cell, such as preMKs, MKs, proplatelets, preplatelets. It also inclusive of “pluripotent stem cells”, which includes embryonic stem cells, embryo-derived stem cells, and induced pluripotent stem cells and other stem cells having the capacity to form cells from all three germ layers of the body, regardless of the method by which the pluripotent stem cells are derived. Pluripotent stem cells are defined functionally as stem cells that can have one or more of the following characteristics: (a) be capable of inducing teratomas when transplanted in immunodeficient (SCID) mice; (b) be capable of differentiating to cell types of all three germ layers (e.g., can differentiate to ectodermal, mesodermal, and endodermal cell types); or (c) express one or more markers of embryonic stem cells (e.g., express October 4, alkaline phosphatase. SSEA-3 surface antigen, SSEA-4 surface antigen, SSEA-5 surface antigen, Nanog, TRA-1-60, TRA-1-81, SOX2, REX1. Progenitor cells also include “megakaryocytic progenitor” (preMK), which refers to a mononuclear hematopoietic cell that is committed to the megakaryocyte lineage and is a precursor to mature megakaryocytes. Megakaryocytic progenitors are normally found in (but not limited to) bone marrow and other hematopoietic locations, but can also be generated from pluripotent stem cells, such as by further differentiation of hemogenic endothelial cells that were themselves derived from pluripotent stem cells.

“PLC” or “PLCs” or artificial platelets as interchangeably used herein, refer to non-naturally existing, novel, anucleated platelets or platelet-like cells that structurally differ from naturally existing bone marrow derived platelets (i.e., natural counterpart). PLC or PLCs are also inclusive of platelet variants, defined elsewhere.

As used herein, the terms “treat,” treating,” “treatment,” and the like refer to reducing or ameliorating a disorder and/or symptom associated therewith. It will be appreciated that, although not precluded, treating a disorder or condition does not require that the disorder, condition or symptoms associated therewith be eliminated.

“Autoantigen” or “self-antigen” as used herein is an antigen (found for example on a receptor or a ligand) that is a normal cellular or bodily constituent and against which the immune system produces autoantibodies. Autoantigen may comprise proteins, nucleic acids, carbohydrates, lipids or various combinations of these that is/are recognized by the immune system of patients suffering from a specific autoimmune disease. Autoantigens may be found in all cell types (e.g. chromatin, centromeres) or be highly specific for a specific cell type in one organ of the body (e.g. thyroglobulin in cells of the thyroid gland).

“Autoantibodies” are antibodies that react with self-antigens. When it is mentioned that an autoantibody binds to a PLC receptor or a ligand, it is generally understood that the autoantibodies are binding to an equivalent of a self-antigen on an endogenous cell against which autoantibodies are generated.

“Donor platelets” refer to bone marrow derived platelets physiologically generated in a mammalian (e.g., human) body.

“Variant” or “Variants” as interchangeably used herein refers to manifesting structural variety, structural deviation, or structural differences between PLCs and donor platelets. As non-limited examples, variant comprises greater than an average of 2%CD63 receptors (i.e., CD63^{>average2%}) as compared to the reference resting bone marrow derived platelet cells with less than an average 2% CD63 receptor i.e., (CD63^{<average 2%}). In some embodiments, a variant comprises less than 10% on an average of CD36 receptor (i.e., CD36^{<average80%}) as compared to the reference resting bone marrow derived platelet cells with greater than an average 80% CD36 receptor i.e., (CD36^{>average 80%}); or a variant comprising less than an average of 95%CD42b receptor (i.e., CD42b^{<average95%}) as compared to the reference resting bone marrow derived platelet cells with greater than an average 95% CD42b receptor i.e., (CD42b^{>average 95%}); or a variant comprising less than an average of 90% glycoprotein VI receptor or less i.e., (GPVI^{<average90%}) as compared to the reference resting bone marrow derived platelet cells with greater than an average 90% GPVI receptor i.e., (GPVI^{>average 90%}). The term variant is also inclusive of a structural makeup of the PLCs that is comparable to the structural make-up of naturally existing bone marrow derived platelets, either in resting or in their activated stages. For example, the PLCs and the donor platelets may have m%CD36, or n%CD42a, or o%CD42a-b-d, or p%CD61, or q%CD62p, or x%CD63 receptors, where the m%, n%, o%, p%, q% x% are the same (i.e., have equal values) between the PLCs and the and bone marrow derived platelets. In other words, structurally PLCs may be identical to donor platelets, yet manifest the advantages of the PLC variants disclosed herein in this application.

The expression “therapeutically effective amount” refers to an amount of the plc or derivatives thereof which is effective for preventing, ameliorating or treating the autoimmune or viral disease in question.

The term “extracellular vesicles (EVs or EV)” as used herein collectively refers to microvesicles and exosomes and generally are very small (generally around 1 micron or less in diameter; microvesicles, generally about 200-1500nm or less in diameter; exosomes generally about 20-200nm or less in diameter) phospholipid vesicle shed from a megakaryocyte or other cell. Extracellular vesicles (EVs) may contain or may transport materials such as but not limited to nucleic acids (e.g., siRNA), growth factors, proteins or exogenous genetic materials (e.g., for gene therapy) and express the extracellular markers of their parental cells. Megakaryocyte-derived extracellular vesicles (EVs) may have a role in multiple pathways, including hemostasis and inflammation, and in treating various disorders, such as but not limited to, malignancies (e.g., neoplasia), Alzheimer, and tumor progression and development.

Abbreviations used herein are disclosed or defined throughout the specification, some are disclosed herein.

EVs, extracellular vesicles; WNV, West Nile virus; hMPV, human metapneuomovirus; FMDV, foot-and-mouth disease virus; HSV, herpes simplex virus; CAR, coxsackievirus and adenovirus receptor; CVB, coxsackievirus B; SV40, Simian virus 40; VP1, viral protein 1; EBOV, Ebola virus; IAV, influenza A virus; MERS-CoV, Middle East Respiratory Syndrome corona virus; HIV, human immunodeficiency virus; BKPyV, BK polyomavirus; SAs, sialic acids; CAMs, cellular adhesion molecules; PtdSer, phosphatidylserine; Neu5Ac, 5-N-acetyl neuraminic acid; HA, hemagglutinin; NA, neuraminidase; RBD, receptor-binding domain; JAM-A, junctional adhesion molecule A; PML, progressive multifocal leukoencephalopathy; PI3K, phosphatidylinositol 3-kinase; IgSF, immunoglobulin superfamily; CAMs, cell adhesion molecules; RGD, arginine-glycine-aspartic acid; IFN, interferon; GPCRs, G-protein coupled receptors; DAF, decay-accelerating factor; JCPyV, JC polyomavirus; LSTc, lactoseries tetrasaccharide c; 5-HT, 5-hydroxytryptamine; IgSF, Immunoglobulin superfamily receptors.

This novel strategy takes advantage of PLC properties, such as, abundant surface receptors, ligands, flexible morphology, circulating properties, cellular signaling, and metabolism (e.g., inducing clearance of autoantibodies or viruses in the liver), to offer a unique opportunity to maximize therapeutic outcomes as well as minimizing side effects in treating autoimmune diseases.

The present disclosure also entails the treatment or prevention of autoimmune diseases by reducing or preventing autoantibodies, associated with the autoimmune diseases, from disrupting the physiological functioning of endogenous cells (e.g., platelets, insulin-producing beta cells in the pancreatic islets etc.). Disruptions to the debilitating effects of the autoantibodies are induced by administering PLCs or derivatives thereof of the present disclosure. The PLCs or their derivatives comprise at least a receptor or a ligand that is commonly shared by the endogenous cells that are recognized by or are targets for the autoantibodies. The receptors or the ligands on the PLCs or their derivatives sequester the autoantibodies by their binding to the PLCs or their derivatives, rather than to the receptors or the ligands (i.e., autoantigens) on the endogenous cells organs or tissues, thereby suppressing the harmful influences of the autoantibodies, when the PLCs or derivatives thereof are administered to a subject. In some embodiments, PLCs or derivatives thereof provide ligands, which act as decoys for the autoantibodies to bind thereto when the shared common element between the PLCs or their derivatives and the endogenous cell population comprises the autoantibody binding ligand.

In some embodiments, the PLC or derivatives thereof of the present disclosure can be generated in a device or a system that supports a biologically active environment e.g., bioreactors or fluidic devices. Bioreactors or fluidic devices could include, but is not limited to, shear stress, mechanical strain and pulsed electromagnetic field bioreactors, large-scale stirred tank bioreactors, automated bioreactors, rotating wall bioreactors (RWBs), and rocking motions as seen with wave bioreactors, organ-on-chip bioreactors. Other bioreactor configurations that enable continuous, perfusion operation such as packed bed bioreactors (PBBs), CultiBag bioreactors, fluidized bed bioreactors (FBBs) and membrane bioreactors such as hollow fiber bioreactors (HFBs) are also contemplated for generating the PLC s/EVs or derivatives thereof of the present invention. Operation of the bioreactors may require coupling with an internal or external cell retention device on a recycle line, by centrifugation, sedimentation, ultrasonic separation or microfiltration with spin-filters, alternating tangential flow (ATF) filtration or tangential flow filtration (TFF) or in-vivo bioreactors, which are a pocket within the body into which biomaterials (e.g., PLCs or their derivatives or the progenitor cells form which they are derived from) are implanted at a site in need thereof and incubated for an extended period of time. Within these pockets (for example, bone tissue or muscle flap etc.), the grafts harness the regenerative capacity of the body to recover from a disease or an injury. Non-limiting examples of bioreactors are described, for example, in the co-filed application titled: Simultaneous Welding of Three Components To Form a Bioreactor or Filter Structure (U.S. Application No. 62/981,373) or elsewhere, for example tools and technologies (e.g., bioreactors or fluidic devices) disclosed in U.S. Pat. Nos. 9,795,965; 10,343,163; 9,763,984; 9,993,503; and 10,426,799; US Publication No. 20180334652; PCT Applications PCT/US2018/021354; PCT/US2019/012437, PCT/US19/040021 and U.S. application Ser. No. 16/730,603, each of which is incorporated herein in their entirely by reference. Bioreactors or microfluidic devices known or unknown that can routinely generate the PLCs or derivatives are also contemplated for use in this invention.

In some embodiments of the present disclosure, the PLCs or their derivatives or the PLC-producing cells (e.g., iPSCs or Megakaryocytes) are genetically engineered to produce PLCs or their derivatives that express one or more antigens on a ligand or a receptor that is shared with an endogenous cell that expresses the same antigen(s) that acts as an autoantigen. The PLC-producing cells (e.g., iPSCs or Megakaryocytes) or the PLCs or their derivatives are genetically engineered by introducing into an isolated population of PLC-producing cells or the PLC population an exogenous nucleic acid. The nucleic of the present disclosure, i.e., a nucleic acid encoding a protein of interest is operably linked to a regulatory element, can be stably inserted into isolated population of PLC-producing cells (e.g., iPSCs or Megakaryocytes) or the PLCs as naked DNA or RNA or more commonly as part of a vector to facilitate manipulation of the nucleic acid. As used herein, the term nucleic acid refers to a nucleic acid molecule (e.g., encoding one or more proteins), which is inserted by artifice into a cell and is stably integrated into the chromosomal genome of the cell or is stably maintained as an episome.

Nucleic acids can be introduced into the isolated population of PLC-producing cells (e.g., iPSCs or Megakaryocytes) or the PLCs or the extracellular vesicles by means of a viral vector. There are many retroviral based vectors. For the present application, the term “retrovirus” includes: murine leukemia virus (MLV), adenovirus, adeno-associated virus, human immunodeficiency virus (HIV), equine infectious anemia virus (EIAV), mouse mammary tumor virus (MMTV), Rous sarcoma virus (RSV), Fujinami sarcoma virus (Fussy), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

### General Structure of the Vectors

Lentiviral vectors are at the forefront of gene delivery systems for research and clinical applications. These vectors can efficiently transduce nondividing and dividing cells, to insert large genetic segment in the host chromatin, and to sustain stable long-term transgene expression. Like other retroviruses, lentiviruses have gag, pol and env genes flanked by two LTR (Long Terminal Repeat) sequences. Each of these genes encodes for numerous proteins which are initially expressed in the form of a single precursor polypeptide. The gag gene encodes for the internal structure proteins (capsids and nucleocapsid). The pol gene encodes for inverse transcriptase, integrase and protease. The env gene encodes for viral envelope glycoprotein. Furthermore, the lentivirus genome contains a cis-acting RRE (Rev Responsive Element) element responsible for exporting out of the nucleus of the viral genomic RNA which will be packaged. The LTR 5′ and 3′ sequences serve to promote the transcription and polyadenylation of the viral RNAs. The LTR contains all the other cis-acting sequences necessary for viral replication. Sequences necessary for the inverse transcription of the genome (linkage site of the RNAt primer) and for the encapsidation of viral RNA in particles (T site) are adjacent to the LTR 5′. If the sequences necessary for encapsulation (or for packaging retroviral RNA in the infectious virions) are absent from the viral genome, genomic RNA will not be actively packaged. Furthermore, the lentiviral genome comprises accessory genes such as vif, vpr, vpu, nef, TAT, REV etc. The construction of lentiviral vectors for gene transfer applications has been described, for example, in patents U.S. Pat. No. 5,665,577, EP 386 882, U.S. Pat. Nos. 5,981,276, 6,013,516 or in patent applications WO99/58701 and WO02/097104. These vectors include a defective lentiviral genome, i.e. in which at least one of the gags, pol and env genes has been inactivated or deleted.

Lentivirus experiments can be performed using lentivirus vectors known to one of skill in the art. As a non-limiting example, lentivirus vectors such as but not limited to GCMV-MCS-IRES-eGFP and GCMV-MCS-IRES-dsRed can be used to deliver a transgene of interest. Both vectors are HIV1 strains that lack the structural viral genes gag, pol, env, rev, tat, vpr, vif, vpu, and nef. In addition, there is a partial deletion of the promoter/enhancer sequences within the 3′ LTR that renders the 5′ LTR/promoter self-inactivating following integration. The genes provided in trans for both vectors are the structural viral proteins Gag, Pol, Rev, and Tat (via plasmid Delta8.9) and the envelope protein VSV-G. These plasmids are introduced into the PLCs, EVs or the PLC-producing progenitor cells by co-transfection, and transiently express the different viral proteins required to generate viral particles. The potential for generating wild type or pathogenic lentivirus is extremely low because it would require multiple recombination events amongst three plasmids. In addition, the virulence factors (vpr, vif, vpu, and nef) have been completely deleted from both vectors.

In some embodiments of the present disclosure, the isolated population of PLC-producing cells (e.g., iPSCs or Megakaryocytes) or the PLCs are genetically engineered by introducing into an isolated population of PLC-producing cells (e.g., iPSCs or Megakaryocytes) or the PLCs a first transgene comprising an inducible promoter and nucleotide sequences encoding one or more exogenous proteins for their transcription under the control of the inducible promoter. Alternatively, a second transgene is introduced into the same platelet population or their progenitor cells, the second transgene comprising a constitutive promoter and a nucleotide sequence encoding a transcription factor for the constitutive expression of the transcription factor under the control of the constitutive promoter, the transcription factor specific for binding to the inducible promoter in the first transgene thereby inducing transcription of the proteins from the coding sequences in the first transgene.

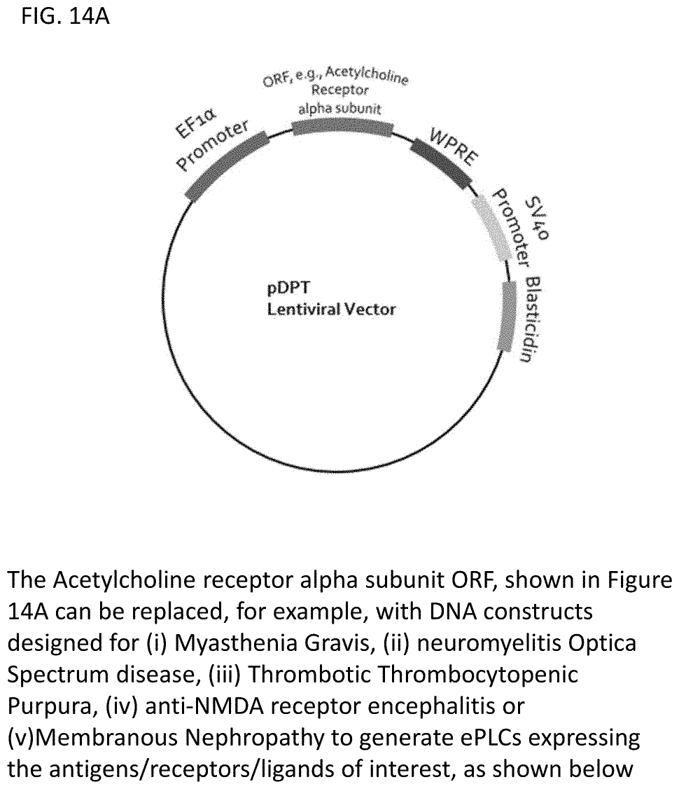

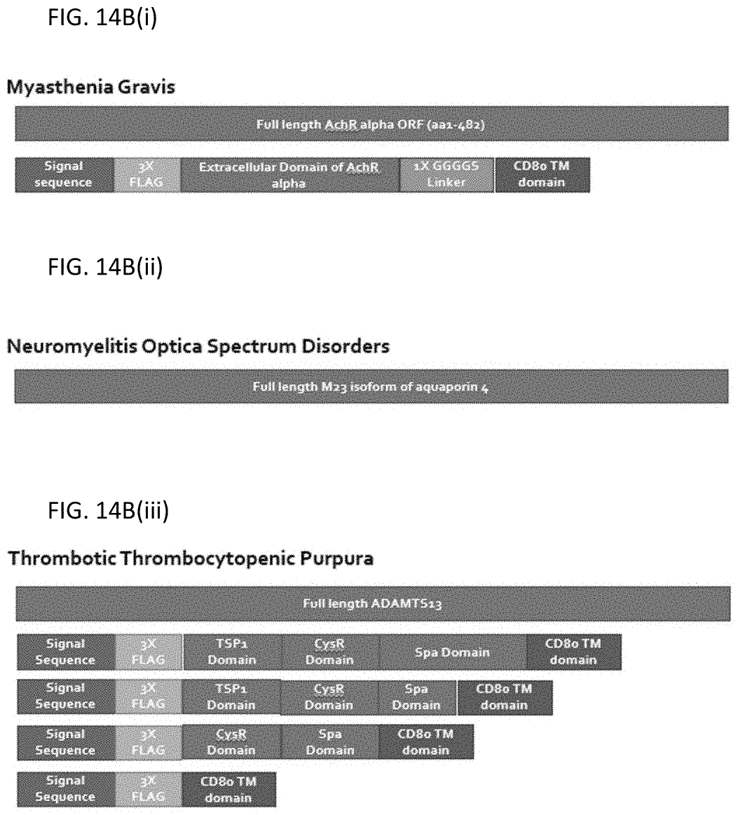

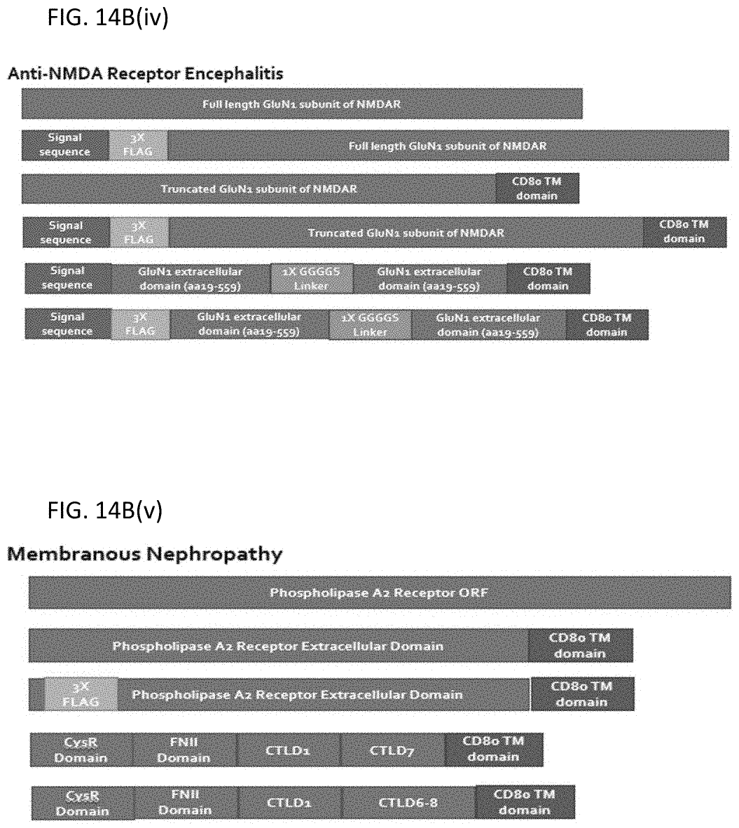

In some embodiments, the exogenous genetic material may be selected from, but not limited to, siRNA, shRNA, ceDNA, DNA, in one or separate vectors with independent inducible (e.g., Tetracycline (Tet) Inducible Expression) or constitutive promoters or a combination thereof. The transgene of the present disclosure, i.e., a nucleic acid encoding a protein of interest (e.g., at least bearing an autoantigen) is operably linked to constitutive or inducible regulatory elements that can be stably inserted into the PLCs or the PLC-producing progenitor cells as naked DNA or more commonly as part of a vector to facilitate manipulation of the transgene. Viral vectors are well known to skill of the art and deposits of such vectors are commercially available at https://www.addgene.org/. Non-limiting, examples of viral vectors and their use to generate ePLCs or the EVs bearing a gene of interest are shown in FIG. 14A and in co-pending U.S. application Ser. No. 17/213,552, incorporated herein by reference in its entirety.

In some embodiments, the step of genetically modifying an expression of a gene in the PLCs comprises modifying a gene of interest with a zinc finger nuclease (ZFN), a Tale-effector domain nuclease (TALEN), or a CRISPR/Cas system. In some embodiments, genetically modifying expression of a gene of interest comprises: i) introducing a clustered regularly interspaced short palindromic repeat-associated (Cas) protein into the PLCs and ii) introducing one or more ribonucleic acids in the PLCs to be modified, wherein the ribonucleic acids direct the Cas protein to hybridize to a target motif of the gene sequence of the gene of interest, and wherein the target motif is cleaved. In some embodiments, the Cas protein is introduced into the PLCs in protein form. In some embodiments, the Cas protein is introduced into the PLCs by introducing a Cas nucleic acid coding sequence. In some embodiments, the Cas protein is Cas9 or a derivative thereof.

In some embodiments, the step of genetically modifying PLC-producing progenitor cells or the PLCs for their gene expression comprises modifying a gene with a zinc finger nuclease (ZFN), a Tale-effector domain nuclease (TALEN), or a CRISPR/Cas system. In some embodiments, genetically modifying PLC-producing progenitor cells or the PLCs for their gene expression comprises: i) introducing a clustered regularly interspaced short palindromic repeat-associated (Cas) protein into the PLC-producing progenitor cells or the PLCs and ii) introducing one or more ribonucleic acids in the PLC-producing progenitor cells or the PLCs to be modified, wherein the ribonucleic acids direct the Cas protein to hybridize to a target motif of a PLC-producing progenitor cells or to a gene sequence in the PLCs, and wherein the target motif is cleaved. In some embodiments, the Cas protein is introduced into the PLC-producing progenitor cells or the PLCs in protein form. In some embodiments, the Cas protein is introduced into the PLC-producing progenitor cells or the PLCs by introducing a Cas nucleic acid coding sequence. In some embodiments, the Cas protein is Cas9 or derivatives thereof. In some embodiments, the target motif is a 10-nucleotide DNA sequence, or 20-nucleotide DNA sequence, or 30-nucleotide DNA sequence, or 40-nucleotide DNA sequence.

In some embodiments are provided modified PLCs comprising a targeted gene alteration that inhibits expression of a gene of interest. In some embodiments, the modified PLCs are generated by disruption of a gene (e.g., a thrombin-related gene). Methods for disrupting a gene of interest include, but are not limited to, methods employing a zinc finger nuclease (ZFN), a Tale-effector domain nuclease (TALEN), and CRISPR/Cas system and an Argonaute protein taken from Pyrococcus furiosus (PfAgo), or a combination thereof. The ribonucleic acids that can direct the Cas protein to and hybridizing to a target motif in the target gene sequence are referred to as single guide RNA (“sgRNA”). The sgRNAs can be selected depending on the particular CRISPR/Cas system employed, and the sequence of the target polynucleotide, as will be appreciated by those skilled in the art. Genetic editing can be accomplished by the well-established gene editing tool of the CRISPR-Cas systems. (Moon, S. B., Kim, D. Y., Ko, J. et al. Recent advances in the CRISPR genome editing tool set. Exp Mol Med 51, 1-11 (2019), incorporated herein in its entirety by reference.

Genetically engineered induced pluripotent stem cells or PSC-derived megakaryocytes that produce the PLCs or derivatives thereof of the present disclosure can be can be generated in a device or a system that supports a biologically active environment e.g., bioreactors or fluidic devices, as discussed in the foregoing. The vector comprising the nucleic acid molecule of interest may be delivered to the cell (e.g., iPS cell, megakaryocytic progenitor, or megakaryocyte) via any method known in the art, including but not limited to transduction, transfection, infection, or electroporation.

In some embodiments of the present disclosure, one or more receptors, ligands or antigens in the PLCs or derivatives thereof may be bioconjugated to a cytotoxic agent, such, as for example, disclosed in co-pending applications PCT/US19/040021 and U.S. application Ser. No. 16/730,603, or co-pending U.S. application Ser. No. 17/213,552, incorporated herein by reference in their entireties. This novel strategy takes advantage of several PLC properties, such as, abundant surface receptors, ligands or antigens, flexible morphology, long circulation time, cellular signaling, and metabolism, to offer a unique opportunity to maximize therapeutic outcomes as well as minimizing side effects. Here, it is envisioned that the PLCs utilize, other than their autoantigenic site(s) to selectively bind to the autoantibodies, their other receptor(s) or ligand(s) to limit the harmful effects of the autoantibodies. For example, the PLC receptors or ligand may be used to conjugate a cytotoxic agent to the PLCs that may facilitate the elimination of the autoantibodies or to deliver a drug to alleviate suffering from autoimmune diseases.

“Bioconjugation” refers to conjugating PLCs or the PLC-producing progenitor cells to a cytotoxic agent with or without the use of a linker. Bioconjugation techniques are well known to one of skill and the art and can be found, for example, in Bioconjugate Techniques, 3rd Edition (2013) by Greg T. Hermanson (ISBN 978-0-12-382239-0: Academic Press). “Bioconjugate Techniques” besides being a complete textbook and protocols-manual for biomolecular crosslinking, it is also an exhaustive and robust reference for conjugation strategies.

### PLCs Receptor for Treating AI Diseases

PLCs are rich in receptors, ligands or antigens on their cell surface or in their transmembrane domain, which makes PLCs an ideal vehicle to trap autoantibodies from binding to autoantigenic endogenous cells or to trap any viral or bacterial proteins or any toxic molecule for their removal and clearance, for example, by the liver. The PLC receptors (exogenous (i.e., genetically expressed) or endogenous to PLCs), that can be utilized to treat or ameliorate autoimmune, antiviral, antibacterial or any other disease in which PLC receptors are involved include, but are not limited to, P2Y1, P2Y12, PAR1, PAR4, Tpa, PAF receptors, PGE2 receptor (EP3), Lysophosphatidic acid receptor, Chemokine receptors, V1a vasopressin receptor, A2a adenosine receptor, b2 adrenergic receptor, Serotonin receptor, Dopamine receptor, P2X1, c-Mpl, Insulin receptor, PDGF receptor, Leptin receptor, GPVI, CD148, CLEC-2, Eph receptor, Axl/Tyro3/Mer, P-selectin, TSSC6, CD151, CD36, TLT-1, PEAR1, VPAC1, PECAM-1, G6B-b, PGI2 receptor (IP), PGD2 receptor, PGE2 receptor (EP4), GPIb-IX-V complex, toll like receptors (TLR 1, TLR 2, TLR 3, TLR 4, TLR 6, TLR 7, TLR 8 or TLR 9), complement receptors, such as but not limited to, CR2, CR3, CR4, C3aR, C5aR, gC1qR and cC1qR, CCL5 and CCL3, or a modified version thereof.

### PLCs Receptor Families for Treating AI Diseases

The PLC receptors discussed in the foregoing belong to one or more the families, such as but not limited to, Leucine-rich repeat family, Ig superfamily, Integrins, Tyrosine phosphatase receptor, C-type lectin receptor, G protein-coupled receptors, Ion channel, Tyrosine kinase receptor, Cytokine, C-type lectin receptor family, tetraspanins, Class B scavenger receptor, Multiple EGF-like domain protein, transmembrane 4 superfamily. Once an autoantigen on an endogenous cell against which autoantibodies are generated is recognized a skilled artisan can manipulate the PLC receptors to block the autoantibodies selecting such receptors from any one member of PLC receptor families. One or more members in each family or in an unrelated family of PLC receptors but sharing the autoantigens with endogenous cells can be used to treat or ameliorate an AI disorder. PLC receptors could be present on the PLC surface, PLC plasma membrane, or in the PLC a granule. In addition to the PLC receptors discussed above, PLC receptors belonging to anyone or more member of the families disclosed herein, as long as they share common autoantigens with endogenous cells, may be used to treat AI, bacterial or viral diseases in accordance with the present disclosure. In a likewise manner, PLCs can be manipulated for trapping and removal of viral, bacterial or toxic molecules, causative agents for diseases.

### PLCs Ligand for Treating AI Diseases

Like the PLC receptors, ligands which makes PLCs an ideal vehicle to trap autoantibodies from binding to autoantigenic endogenous cells or to trap any viral or bacterial proteins or any toxic molecule for their removal and clearance, for example, by the liver are inclusive of, but limited to, vWf, thrombin, FXI, FXII, P-selectin, HK, Mac-1, TSP-1, Collagen, laminin, Fibronectin, Vitronectin, fibrinogen, vWf, osteopontin, fibrin, vWf, TSP-1, Podoplanin, ADP, Thrombin, Thromboxane, 1-O-alkyl-2-acetyl-sn-glycero-3-phosphocholine, PGE2, Lysophosphatidic acid, Chemokines, Vasopressin, Adenosine, Epinephrine, Serotonin (5-hydroxytryptamin), Dopamine, ATP, TPO, Insulin, PDGF, Leptin, Ephrin, Gas-6, PSGL-1, GPIb, TF, TSP1, oxLDL, VLDL, oxPL, collagen type V, Fibrinogen, PACAP, PECAM-1, collagen, glycosaminoglycans, PGI2, PGD2.

### Extracellular Vesicles (EV)

In some embodiments, the present disclosure comprises microvesicles and exosomes (collectively referred to as extracellular vesicles (EVs)) or derivatives thereof, which are produced admixtures of the PLCs. Given that EVs or derivatives thereof carry receptors, bioactive lipids, nucleic acids, such as mRNA and microRNA (miRNA) or siRNA, proteins, they are able to deliver important payloads to recipient cells (e.g., tumor cells or to an organ or tissue such as liver).

EVs or derivatives thereof of the present disclosure can be isolated and purified, essentially separating them from an admixture comprising the PLCs of the invention. Isolated or purified extracellular vesicles (EVs) or derivatives thereof, because of their ability to extensively travel throughout the body, can exert remarkable therapeutic effects when administered to a patient on their own. EVs have a fundamental immunomodulatory potential for treating or inhibiting inflammatory diseases. EVs could also be used as drug delivery system; they are able to cross biological barriers, including the blood-brain barrier and synovial membrane.

Advantageously, EVs or derivatives thereof can be internalized by recipient cells following receptor-ligand interactions and the varied assortment of bioactive molecules, derived from the cell of origin, such as proteins, bioactive lipids, and nucleic acids, can be transferred along with the proteins expressed on the EV surface.

In some embodiments, EVs or derivatives thereof may directly activate a recipient cell (e.g., donor platelets) by acting as signaling complexes. For example, EVs or derivatives thereof may bind to platelets by means of the P-selectin glycoprotein ligand-1 expressed on their surface and EVs or derivatives thereof from neutrophils expressing Mac-1 may induce donor platelet activation in a patient in need thereof.

Compositions and methods comprising the extracellular vesicles (EV) or derivatives thereof of the present disclosure can be used in several therapies or co-therapies, such as for treating or inhibiting inflammatory diseases, delivery of genes, proteins or peptides, nucleic acids for the use in cellular or gene therapies, for example using vectors, e.g. adenovirus, lentivirus, to obtain novel microvesicle or exosomal gene (e.g., for gene therapy), peptide (for growth factors) or nucleic acid (e.g., siRNA or microRNA) delivery vehicles. Packaging within extracellular vesicles (EV) provides several advantages such as shielding the molecules from adverse cellular event that may neutralize the naked gene. Engineered extracellular vesicles (EV) could be used to carry drugs to specific sites of tissue damage, including but not limited to cancer, Alzheimer and other disorders discussed elsewhere herein.

The extracellular vesicles (EV) or derivatives thereof of the present disclosure are isolated as described in the experimental section of the description. The isolated extracellular vesicles (EV) derivatives thereof may then be stored until use by freezing at very low temperature, e.g. at −80° C. in presence of cryopreserving agents, such dimethylsulphoxide (DMSO) and glycerol used at optimal concentrations.

In some embodiments, an average diameter of extracellular vesicles (EV) derived from a population of iPSC derived platelets is less than 50% the diameter of the extracellular vesicles (EV) derived from a population of donor derived platelets having about the same number of platelets as the population of iPSC derived platelets. In some embodiments, the megakaryocyte or platelet is genetically modified to comprise a nucleic acid molecule encoding a therapeutic agent.

Extracellular vesicles (EV) are subcellular sized particles consisting of a membrane lipid bilayer and cellular content. Extracellular vesicles (EV) isolated or purified from an admixture comprising PLCs may exert both anti-inflammatory and pro-inflammatory function and have potential as vehicles for drug delivery.

In some embodiments, the diameter of the instant extracellular vesicles (EV) is 0.1 and 4 μm. In some embodiments, the diameter of the instant extracellular vesicles (EV) is 0.1 and 3 μm. In some embodiments, the diameter of the instant extracellular vesicles (EV) is 0.1 and 2.5 μm. In some embodiments, the diameter of the instant extracellular vesicles (EV) is 0.1 and 2 μm. In some embodiments, the diameter of the instant extracellular vesicles (EV) is 0.1 and 1.5 μm. In some embodiments, the diameter of the instant extracellular vesicles (EV) is 0.1 and 1.0 μm. In some embodiments, the diameter of the instant extracellular vesicles (EV) is 0.1 and 0.9 μm. In some embodiments, the diameter of the instant extracellular vesicles (EV) is 0.1 and 0.8 μm. In some embodiments, the diameter of the instant extracellular vesicles (EV) is 0.1 and 0.7 μm. In some embodiments, the diameter of the instant extracellular vesicles (EV) is 0.1 and 0.6 μm. In some embodiments, the diameter of the instant extracellular vesicles (EV) is 0.1 and 0.5 μm. In some embodiments, the diameter of the instant extracellular vesicles (EV) is 0.1 and 0.4 μm. In some embodiments, the diameter of the instant extracellular vesicles (EV) is 0.1 and 0.3 μm. In some embodiments, the diameter of the instant extracellular vesicles (EV) is 0.1 and 0.2 μm. In some embodiments, the diameter of the instant extracellular vesicles (EV) is 0.2 and 1 μm. In some embodiments, the diameter of the instant extracellular vesicles (EV) is 0.3 and 1 μm. In some embodiments, the diameter of the instant extracellular vesicles (EV) is 0.4 and 1 μm. In some embodiments, the diameter of the instant extracellular vesicles (EV) is 0.5 and 1 μm. In some embodiments, the diameter of the instant extracellular vesicles (EV) is 0.6 and 1 μm. In some embodiments, the diameter of the instant extracellular vesicles (EV) is 0.7 and 1 μm. In some embodiments, the diameter of the instant extracellular vesicles (EV) is 0.8 and 1 μm. In some embodiments, the diameter of the instant extracellular vesicles (EV) is 0.9 and 1 μm. In some embodiments, the diameter of the instant extracellular vesicles (EV) is 0.2 and 2 μm. In some embodiments, the diameter of the instant extracellular vesicles (EV) is 0.3 and 2 μm. In some embodiments, the diameter of the instant extracellular vesicles (EV) is 0.4 and 2 μm. In some embodiments, the diameter of the instant extracellular vesicles (EV) is 0.5 and 2 μm. In some embodiments, the diameter of the instant extracellular vesicles (EV) is 0.6 and 2 μm. In some embodiments, the diameter of the instant extracellular vesicles (EV) is 0.7 and 2 μm. In some embodiments, the diameter of the instant extracellular vesicles (EV) is 0.8 and 2 μm. In some embodiments, the diameter of the instant extracellular vesicles (EV) is 0.9 and 2 μm. In some embodiments, the diameter of the instant extracellular vesicles (EV) is 1.0 and 2 μm. In some embodiments, the diameter of the instant extracellular vesicles (EV) is 1.5 and 2 μm. In some embodiments, the diameter of the instant extracellular vesicles (EV) is 2.0 and 2.5 μm.

Extracellular vesicles (EV) derivatives thereof may be conjugated to one or more cytotoxic agents by mechanisms disclosed in the foregoing. One or more cytotoxic agents may be imbibed into the extracellular vesicles (EV) derivatives thereof by mechanisms also disclosed in the foregoing. Cytotoxic agents are also disclosed in the foregoing. Diseases and disorders that can be cured or mitigated by the use of EVs derivatives thereof alone or in combination with the PLCs or derivatives thereof of the present disclosure are also disclosed below.

In some embodiments, EVs, whether modified or not (e.g., bioengineered or conjugated) may be developed for therapeutic use independent of the PLCs or derivatives therefrom. For example, a patient in need of a treatment predominantly involving microvesicles or derivatives thereof will be administered microvesicle-based treatment or exosome-based treatment or a combination of both. For example, MVs or exosomes incorporated with exogenous siRNAs can be used for efficient silencing of a target MAPK gene in monocytes and lymphocytes or deliver VEGF-siRNA across the blood-brain barrier or siRNAs targeting Huntingtin disease, or siRNA into the liver, among others. Advantageously, MVs could be used as more efficient delivery vehicles to direct specific targeting of novel therapeutics without immunogenicity and adverse effects. In some embodiments, EVs, for example exosomes, may be pulsed with tumor peptides to make cell-free cancer vaccines.

In some embodiments, the EV-based treatment may be administered prior to PLC-based treatment. In some embodiments, PLC-based treatment may be administered prior to EV-based treatment. In some embodiments, PLCs and EVs are administered as admixtures. Also contemplated are treatments in which admixtures comprising PLCs and EVs are administered followed by treatment regiments comprising essentially of EV or derivatives thereof or comprising essentially of PLCs or derivatives thereof-based treatment depending on a patient's need.

Co-Therapy with Therapeutic Agents

Concomitant with the use of the PLCs or derivatives thereof to treat AI, viral, bacterial or other diseases, other agents that can be co-administered or administered separately to treat autoimmune viral, bacterial or other diseases include antibodies and drugs. When an antibody is selected, the antibody may be raised against antigens such as but not limited to α-subunit of AChR, Aquaporin-4, ADAMTS13, NMDAR, phospholipase A2R, renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-.beta.; platelet-derived growth factor (PDGF); fibroblast growth factor such as aFGF and bFGF; fibroblast growth factor receptor 2 (FGFR2), epidermal growth factor (EGF); transforming growth factor (TGF) such as TGF-alpha and TGF-beta, including TGF-.beta.1, TGF-.beta.2, TGF-.beta.3, TGF-.beta.4, or TGF-.beta.5; insulin-like growth factor-I and -II (IGF-I and IGF-II); des(1-3)-IGF-I (brain IGF-I), insulin-like growth factor binding proteins, EpCAM, GD3, FLT3, PSMA, PSCA, MUC1, MUC16, STEAP, CEA, TENB2, EphA receptors, EphB receptors, folate receptor, FOLR1, mesothelin, cripto, alphavbeta6, integrins, VEGF, VEGFR, EGFR, tarnsferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152, IFN gamma TNF alpha, IFN alpha, GM-CSF, IL-3. or an antibody which binds to one or more tumor-associated antigens or cell-surface receptors; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-2, IL-6, IL-12, IL-23, IL-12/23 p40, IL-17, IL-15, IL-21, IL-1a, IL-1b, IL-18, IL-8, IL-4, IL-3, and IL-5; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3 or HER4 receptor; endoglin, c-Met, c-kit, 1GF1R, PSGR, NGEP, PSMA, PSCA, LGR5, B7H4, TAG72 (tumor-associated glycoprotein 72) and fragments of any of the above-listed polypeptides, may be used for a co-therapy.

In some embodiments, the cytotoxic agents are optionally antibodies per se. Antibodies that can be used with the PLCs or derivatives thereof include, but are not limited to, antiFcRn antibody (e.g., Efgartigimod, Nipocalimab, Rozanolixizumab, RVT-1401), abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ranibizumab, abciximab, raxibacumab, caplacizumab, infliximab, bevacizumab, dabigatran, Idarucizumab, or ustekinumab (Stelara) or a combination thereof. Furthermore, the antibodies may be selected from anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD1-antibody, anti-CD11a antibody , anti-CD11c antibody, anti-CD13 antibody, anti-CD14 antibody, anti-CD15 antibody, anti-CD18 antibodies; anti-L3 T4 antibodies, anti-CD19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD39 antibody, anti-CD41 antibody, anti-LCA/CD45 antibody, anti-CD45RO antibody, anti-CD45RA antibody, anti-CD71 antibody, anti-CD95/Fas antibody, anti-CD99 antibody, anti-CD100 antibody, anti-S-100 antibody, anti-CD106 antibody, anti-ubiquitin antibody, anti-c-myc antibody, anti-cytokeratin antibody, anti-lambda light chains antibody, anti-melanosomes antibody, anti-prostate specific antigen antibody, anti-tau antigen antibody, anti-fibrin antibody, anti-keratins antibody, and anti-Tn-antigen antibody, heterologous anti-lymphocyte globulin; pan-T antibodies, preferably anti-CD3 or anti-CD4/CD4a antibodies; soluble peptide containing a LFA-3 binding domain or a combination thereof.

In either of the methods described herein, the patient may also optionally be administered one or more additional compounds e.g., glucocorticosteroids, e.g., prednisone, methylprednisolone, or dexamethasone, a glucocorticoid receptor modulator, NSAID, COX-2 inhibitor, DMARD, biologic, small molecule immunomodulator, xanthine, anticholinergic compound, beta receptor agonist, bronchodilator, non-steroidal immunophilin-dependent immunosuppressant, vitamin D analog, psoralen, retinoid, or 5-amino salicylic acid, corticosteroids including, for example, dexamethasone, betamethasone, triamcinolone, triamcinolone acetonide, triamcinolone diacetate, triamcinolone hexacetonide, beclomethasone, dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, and triamcinolone acetonide 21-palmitate, 2-amino-6-aryl-5-substituted pyrimidines, azathioprine; cyclophosphamide; bromocryptine; danazol; dapsone; glutaraldehyde, anti-idiotypic antibodies for MHC antigens and MHC fragments; cyclosporin anti-idiotypic antibodies for MHC antigens and MHC fragments; cyclosporin A, streptokinase; TGF-.beta.; streptodornase; RNA or DNA from the host; FK506; RS-61443; deoxyspergualin; rapamycin; T-cell receptor, A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495.times.(GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG) or combinations thereof.

### Autoimmune Diseases

Autoimmune disease that can be treated by the embodiments of the present disclosure include but are not limited to, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Neuromyelitis Optica Spectrum Disorder, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, Membranous Nephropathy, IgG4-related sclerosing disease, Immune thrombocytopenic purpura (ITP), acquired thrombotic thrombocytopenic purpura (aTTP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neuromyelitis Optica Spectrum Disorder, Neutropenia, N-methyl D-aspartate (NMDA) receptor (NMDAR) Encephalitis, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), acquired thrombotic thrombocytopenic purpura (aTTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease. Autoimmune diseases or disorders also include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitis); multiple sclerosis; Reynaud's syndrome; autoimmune thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; autoimmune polyendocrinopathies; Reiter's disease; stiff-man syndrome; Behcet disease; giant cell arteritis; immune complex nephritis; IgM polyneuropathies; autoimmune thrombocytopenia or any other autoimmune disease as long as the cells that are target of autoimmune antibodies share a common autoantibody binding receptor, ligand or an antigen with the PLCs or derivatives thereof.

### Viral Infections and Treatments

The hallmarks associated with viral infections are many. For example, COVID-19 disease includes the development of respiratory symptoms with significant risk of progressing to respiratory failure and acute respiratory distress syndrome (ARDS), the major cause of mortality. In addition to the respiratory system, viremia, and other organ involvement, including myocarditis, have been documented reflecting systemic involvement of the disease. Full-genome sequencing and phylogenic analysis indicate that the corona virus that causes COVID-19 is a betacoronavirus, found in the same subgenus as the severe acute respiratory syndrome virus (SARS-CoV1), which utilizes the same protein on the cell surface, ACE2, for cell entry. The importance of ACE2 for SARS-CoV2 infection is further illustrated by reports of soluble exogenous ACE2 protein interfering with SARS-CoV2 infection in-vitro.

SARS-CoV-2, a single-stranded RNA-enveloped virus, targets cells through the viral structural spike (S) protein that binds to the angiotensin-converting enzyme 2 (ACE2) receptor. Thus, in some embodiments, the present disclosure provides platelet variants (henceforth referred to as platelet-like cells or PLCs) that feature the enhanced capability of neutralizing coronaviruses, such as SARS-CoV2, through expression of the angiotensin-converting enzyme 2 (ACE2) on the PLC cell surface.

Thus, in some embodiments, the present disclosure provides a genetically engineered PLC designed to overexpress ACE2 (referred to as ACE2-PLC) as an allogeneic cell therapeutic agent against current and future SARS-CoV or SARS-CoV-related infections. In some embodiments, PLCs will act as “decoys” that express rhACE2 on its surface, causing virus binding, internalization, and rapid clearance of the infected PLCs through the liver and spleen, thus removing the infectious viral particles from circulation. Likewise, the PLCs could be genetically engineered to express one or more receptors which interferes with viral replication or integration. Such receptors could be specific to viral membrane (M) protein, neucocapsid (N) protein and viral RNA, or the viral envelop (E) protein. SARS-CoV-2 is known to have four structural proteins: The E and M proteins, which form the viral envelope; the N protein, which binds to the virus's RNA genome; and the S protein, which binds to human receptors. The viral genome consists of more than 29,000 bases and encodes at least 29 proteins. The nonstructural proteins get expressed as two long polypeptides, the longer of which gets chopped up by the virus's main protease. This group of proteins includes the main protease (Nsp5) and RNA polymerase (Nsp12). It is envisioned that the PLCs can be genetically engineered to interfere with any of these viral proteins.

In some embodiments, the genetically engineered PLCs further utilize endogenous receptors, such as but not limited to CD147, to bind to and neutralize viruses (e.g., SARS-CoV2) to promote viral elimination by phagocytosis and subsequent clearance by other immune cells, such as but not limited to, macrophages, natural killer cells or neutrophils.

In some embodiments, the exogenously expressed PLC receptors (e.g., ACE2) or the PLC endogenous receptors (e.g., CD147) bind to and neutralize viruses (e.g., SARS-CoV2) by promoting viral elimination by phagocytosis and subsequent clearance by other immune cells, such as but not limited to, macrophages, natural killer cells or neutrophils. For example, T cells effect clearance of persistent and many acute virus infections via production of antiviral cytokines such as tumor necrosis factor-α and interferon-γ.

In some embodiments, the surface molecules of the PLCs can involve different partners, for example, viral proteins that bind host glycans, receptor proteins, adhesion proteins or peptidase or they could be glycans that bind host lectins or they could viral lipids that bind host receptor proteins. Viruses or viral receptors that may be targeted with the genetically modified PLCs or the PLCs of the present disclosure include, adhesion or entry receptors (i.e., receptors that trigger virus entry) on dsDNA viruses, ssDNA viruses, dsRNA viruses, ssRNA(+) viruses, ssRNA(−) viruses, ssRNA(RT) viruses, or dsRNA(RT) viruses.

Non-limiting examples of viruses, the protein of which could be targeted by the genetically engineered PLCs or the PLCs, include severe acute respiratory syndrome or Middle East respiratory syndrome corona virus(MERS), severe acute respiratory syndrome, also referred to as corona virus disease 2019 (COVID-19), caused by severe acute respiratory syndrome corona virus 2 (SARS-CoV-2) herpes simplex virus (HSV), such as HSV-1 and HSV-2, varicella zoster virus (VZV), Epstein-Barr virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses, the protein of which could be targeted by the genetically engineered PLCs or the PLCs, include, but not limited to, Betacoronavirus, Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae; lentiviruses (e.g., HTLV-I; HTLV-II; HIV-1 (also known as HTLV-III, LAV, ARV, hTLR, etc.) HIV-II); simian immunodeficiency virus (SIV), human papillomavirus (HPV), influenza virus and the tick-borne encephalitis viruses.

PLC receptors (native or exogenously expressed) that specifically engage with specific viruses can be used to neutralize DNA or RNA-based viruses such that the viral elimination occurs by phagocytosis, subsequently leading to viral clearance by immune cells, such as but not limited to, macrophages, natural killer cells or neutrophils or by other B cell or T-cell-based viral clearance mechanisms. Non limiting examples of PLC surface receptors (native or exogenously expressed) include αvβ3, αIIbβ3, a 2β1, Dendritic Cell-Specific Intercellular adhesion molecule-3-Grabbing Non-integrin (DC-SIGN), GP-VI, CR2, α2β1, α2β3 (GPIIbIIIa), Sialic acids, Glycosaminoglycans, ACE2, IgSF member CAR, decay-accelerating factor (DAF) (CD55), serotonin 5-HT₂Rs, α2,6-LSTc, PtdSer, PtdSer-mediated virus entry enhancing receptors PVEERs, cell immunoglobulin and mucin domain (TIM), TYRO3, AXL, MER Proto-Oncogene, Tyrosine Kinase (MERTK) family of receptor tyrosine kinases (Tyro-Axl-MerTK; TAMs), Niemann-Pick C1 receptor, PVEERS, CD4, CCR4, CXCR5, α4β7 integrin, selectins, cadherins, integrins, or SA, which are adhesion or entry receptors for Viruses, such as but not limited to Hantaviruses, Coxackievirus A9, A1; Adenovirus type 2, Echovirus 9, Parechovirus, Hantaviruses, Echovirus 1, Rotavirus, Lentiviruses, HIV; Ebola virus, LASV, LASV, HCV, EBV, Rotavirus, Adenovirus, Influenza, RSV. In some embodiments, it is envisioned that the concentration of endogenous viral adhesion or entry receptors can be enhanced by genetically engineering the PLCs. For example, TLR receptors can be overexpressed in the PLCs by genetically engineering the PLCs such that greater number of receptors are available to interact with the viruses. Stated another way, if PLCs express X% of TLR receptors, by genetically engineering the PLCs, PLCs now express Y% of TLR receptors where Y% is greater that X%.

In some embodiments, the PLCs are genetically engineered to overexpress the PLC receptors to facilitate the clearance of viruses. For example, genetically engineered PLCs overexpress α2bβ3 (GPIIbIIIa) which would facilitate the clearance of adenoviruses. In some embodiments, CR3 or CR5 may be overexpressed in the PLCs to expeditiously suppress viral activities.

In some embodiments, the genetically engineered PLCs, expressing or overexpressing a specific viral binding protein (e.g., ACE2 or alpha v beta 3) may act as a decoy for the viruses to be tricked into binding thereto. Once engulfed by the genetically modified PLCs, the virus is phagocytized by the immune system. For example, ACE2 expressing PLCs will trick COVID-19 virus to bind to it subsequently leading to the viral destruction by the immune system. In this aspect, it is contemplated that the genetically modified PLCs act as immune phagocytes. Phagocytosis is an elegant process for the ingestion and elimination of pathogens by the PLCs.

Endogenous PLC receptors may also act as a decoy for viruses to be tricked into binding thereto. In some embodiments, such endogenous receptors are introduced as a transgene to supplement the endogenous receptors thereby increasing their concentration. Non-limiting examples of such receptors include alpha v beta 3 (αvβ3) or alpha v beta 5 (αvβ5), which interact with adenoviruses, and PLC glycol proteins GPIa/IIa (α2β1 integrin) and GPVI (a member of the Ig super family and primary signaling receptor for platelet activation by collagen), which are known to bind viruses. PLCs also express Coxsackie-Adeno receptor (CAR). This receptor is important for the interaction of Coxsackieviruses B (CVB) with PLCs.

In some embodiments, the PLC-producing cells (e.g., iPSCs or Megakaryocytes) are genetically engineered to produce PLCs expressing a receptor which is a specific target for a viral, such as but not limited ACE2, receptor. The PLC-producing cells (e.g., iPSCs or Megakaryocytes) or the PLCs are genetically engineered by introducing into an isolated population of PLC-producing cells or the PLC population an exogenous nucleic acid encoding a viral interacting receptor (e.g., ACE2). In some embodiments, the nucleic of the present disclosure, i.e., a nucleic acid encoding a protein of interest is operably linked to a regulatory element, can be stably inserted into isolated population of PLC-producing cells (e.g., iPSCs or Megakaryocytes) or the PLCs as naked DNA or RNA or more commonly as part of a vector to facilitate manipulation of the nucleic acid. As used herein, the term nucleic acid refers to a nucleic acid molecule (e.g., encoding one or more proteins), which is inserted by artifice into a cell and is stably integrated into the chromosomal genome of the cell or is stably maintained as an episome.

Nucleic acids can be introduced into the isolated population of PLC-producing cells (e.g., iPSCs or Megakaryocytes) or the PLC by means of a viral vector, such as but not limited to adenoviral vectors, adeno-associated viral vectors, herpes simplex viral vectors, vaccinia viral vectors, baculoviral vectors or retroviral vectors. There are many retroviral based vectors. For the present application, the term “retrovirus” includes: murine leukemia virus (MLV), human immunodeficiency virus (HIV), equine infectious anemia virus (EIAV), mouse mammary tumor virus (MMTV), Rous sarcoma virus (RSV), Fujinami sarcoma virus (Fussy), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), adenoviral vectors, adeno-associated virus (AAV), and Avian erythroblastosis virus (AEV) and all other retroviridiae including lentiviruses.

### Method of Diagnosis and Diagnostic Kit.

The present disclosure provides a diagnostic method comprising: (a) obtaining a sample from an individual (e.g., an asymptomatic individual or a patient); (b) admixing with said sample a composition comprising at least one anti-viral agent (for example PLCs or variants thereof that exogenously or endogenously express a viral adhesion or entry receptor protein) (c) determining the presence or absence of a virus or viral particles or viral peptides or viral nucleic acids or a combination thereof in the sample.

The present disclosure also provides a method of screening for a virus, which comprises: (a) obtaining a sample from an individual (e.g., an asymptomatic individual or a patient); (b) admixing with the sample a composition comprising an anti-viral agent (for example PLCs or variants thereof that exogenously or endogenously express a viral entry receptor protein); (c) determining the presence or absence of a virus or viral particles or viral peptides or viral nucleic acids or a combination thereof in the sample.

The present disclosure also provides a method of screening for a virus, the method comprising: (a) obtaining a sample from an individual ((e.g., an asymptomatic individual or a patient); (b) admixing with the sample a composition comprising an anti-viral agent (e.g., for example PLCs or variants thereof that exogenously or endogenously express a viral entry receptor protein) (c) determining the presence or absence of a virus or viral proteins in the sample by assessing the viral reverse transcription activity in the sample, optionally wherein the viral reverse transcription activity is determined by direct contact of the sample with synthetic RNA, unlabeled and or labeled probes.

The present disclosure also provides a method of screening for a virus, the method comprising: (a) obtaining a sample from an individual (e.g., an asymptomatic individual or a patient); (b) admixing with said sample a composition comprising an anti-viral agent (for example PLCs or variants thereof that exogenously or endogenously express a viral entry receptor protein) (c) determining the presence or absence of a virus or viral proteins in the sample by assessing the viral protease activity in the sample, optionally where the viral protease activity may be assesses by directly contacting the sample with unlabeled or labeled probes containing cleavage sites for viral protease.

The present disclosure also provides a method of screening for a virus, said method comprising: (a) obtaining a sample from an individual (e.g., an asymptomatic individual or a patient); (b) admixing with said sample a composition comprising an anti-viral agent (for example PLCs or variants thereof that exogenously or endogenously express a viral entry receptor protein) (c) determining the presence or absence of virus or viral proteins in the sample by assessing viral replication, optionally the viral replication may be determined after contacting the sample with the PLCs.

In some embodiments, the virus may be resistant to an antiviral medicament. The virus may be resistant to an anti-viral agent, for example an anti-retroviral agent but can be treated by the embodiments of the present disclosure. In some embodiments, PLCs or derivatives thereof may be co-administered with antiviral compounds such as but not limited to Acyclovir, Valacyclovir, Cidofovir, Foscarnet, Ganciclovir, Valganciclovir, Penciclovir, Famciclovir, Idoxuridine, Trifluorothymidine, Vidarabine, Fomivirsen, Amantadine, Rimantadine, Zanamivir, Oseltamivir, Ribavirin, Interferons, Lamivudine, Adefovir, Dipivoxil, Entecavir, Telbivudine, Clevudine, elvucitabine, valtorcitabine, amdoxovir, racivir, MIV 210, β-1-FddC, alamifovir and hepavir B, Imiquimod, Podophyllotoxin, Pleconaril, Maraviroc, Enfuvirtide, Zidovudine, Didanosine, Zalcitabine, Stavudine, Lamivudine, Abacavir, Emtricitabine, Tenofovir disoproxil fumarate, Nevirapine, Delavirdine, Efavirenz, Raltegravir, Saquinavir, Indinavir, Ritonavir, Nelfinavir, Amprenavir, Fosamprenavir, Lopinavir, Atazanavir, Tipranavir, Darunavir, 2′,3′-didehydro-3′-deoxy-4′-ethynylthymidine, MIV-310, SPD754, and L-d4FC or any other drug known to have potent antiviral activity.

The present disclosure also provides a method for identifying a viral infection in a mammal (e., dog, horse, cats, pigs, cows etc.) using any of the methods or diagnostic methods described herein.

The present disclosure also provides method for controlling a viral infection in mammals (e.g., human who are positive for COVID-19) comprising the identification of a viral infection in a mammal using any of the methods or diagnostic methods described herein and, optionally, the isolation of virus-infected mammals from other mammals.

The present disclosure provides a kit comprising at least one anti-viral agent comprising at least the PLCs or variants thereof that exogenously or endogenously express a viral entry receptor protein for use according to the invention, and optionally instructions for administration to said mammal.

In some embodiments, the kit is a diagnostic kit. Such kits may be useful in the diagnosis of a viral infection in a mammal. By using a kit according to the present disclosure or by using a diagnostic method of the present invention, mammal that are infected with a virus, such as COVID-19 or variants thereof can be identified.

Patients who test positive for the virus, for example COVID-19, may be isolated from non-infected population (e.g., a healthy individual which do not have the virus). Advantageously, this helps to prevent the spread of viral infection from infected population to those which are not infected, thereby controlling viral infection within a population.

Patients with viral infection may be monitored by using a kit according to the present disclosure or by using a diagnostic method of the present disclosure to determine the progression of the viral infection or determine the progression of viral disease on a daily or a weekly basis. For example, patients with a viral infection may be isolated and quarantined from non-infected population once the level of infection and/or the progression of the virus have reached a critical point. Typically, patients should be isolated prior to or as soon as patients are detected positive for the viral infection. A healthy individual that is individual who are not infected by a virus should be vaccinated by known or any future virus vaccine that may be developed. Existing viral vaccines are disclosed in chapter 36, titled: Viral Vaccines in Review of Medical Microbiology and Immunology; 14^{th} Edition, published by McGraw-Hill) or developed by technologies disclosed in New Vaccine Technologies to Combat Outbreak Situations Front. (Immunol., 19 Sep. 2018 https://doi.org/10.3389/fimmu.2018.01963) or any successful vaccine that is being developed to fight COVID-19 and COVID-19-related viruses (Developing Covid-19 Vaccines at Pandemic Speed; New England Journal of Medicine Mar. 30, 2020-DOI: 10.1056/NEJMp2005630).

In addition to the agents of the invention, the kits and diagnostic methods of the present disclosure may include, but are not limited to, the following techniques; competitive and noncompetitive assays, radioimmunoassay, bioluminescence and chemiluminescence assays, fluorometric assays, infrared assays, sandwich assays, immunoradiometric assays, dot blots, enzyme linked assays including ELISA, microtiter plates, antibody coated strips, or dipsticks for rapid monitoring of urine or blood, and immunocytochemistry, DNA or RNA amplification techniques including polymerase chain reaction, reverse transcription and LAMP assays. For each kit the range, sensitivity, precision, reliability, specificity, and reproducibility of the assay are established. Intra-assay as well as inter-assay variation may be established at 20%, 50% and 80% points on the standard curves of displacement or activity.

Measurement of Viral Proteins and Nucleic Acid may be carried out by, for example, Polymerase Chain Reaction (PCR), immunoblotting, Immunoprecipitation, Enzyme-Linked Immunosorbent Assays (ELISA), Hemagglutination Assay (HA). Direct Counting of Viral Particles can be carried out with Flow Cytometry (FCM) based viral quantification, Transmission Electron Microscopy (TEM), Immunofluorescence Foci Assay (IFA) or other techniques that may be developed for rapid viral quantification.

In some embodiments, one or more reagents or the receptors, ligands or antigens with which the reagents interact with or the viral adhesion or entry receptors on the cell surface of the PLCs are labeled. The label is selected from the group consisting of a radiolabel, a fluorophore, a chromophore, an imaging agent and a metal ion. Labeling techniques are well known to one of skill in the art.

The kits are readily available to an individual in need thereof. The kit can be used by or applied to a mammal, inclusive of human, cats, dogs, equine, porcine or any mammal in which viral infections are not an uncommon event.

The sample may be blood, blood serum, plasma, saliva, sputum, urine, fecal biopsy, lymph node biopsy, milk, semen, tear drops or sweat or from the proximity of an organ which are rich in viral adhesion or entry receptors. The diagnostic procedures are typically carried out in-vitro or ex vivo.

Once an individual is diagnosed or screened and identified as a patient that is infected by a virus the patient may be immediately put on another antiviral medication as determined by a practitioner. Viral medications are well known to one of skill in the art and can be selected from any of the known anti-viral medications or future anti-viral medications. A list of commonly used anti-viral medications can be found in publication such as “Antiviral Drugs for Viruses Other Than Human Immunodeficiency Virus” (Mayo Clin Proc. 2011 Oct; 86(10):1009-26. doi: 10.4065/mcp.2011.0309 or in “Approved Antiviral Drugs over the Past 50 Years” reviewed in Clinical Microbiology Reviews June 2016, 29 (3) 695-747; DOI: 10.1128/CMR.00102-15).

The scavenging effect of the receptors, ligands or antigens on the PLCs, whether endogenous to PLCs or exogenously expressed in the PLCs (i.e., genetically engineered into the PLCs) is not limited to autoantibodies or viruses but also applies to clearance of bacteria (e.g., PLC-bound listeria; PLC-bound bacteria eventually bind to CRIg on Kupffer cells (in liver) and are phagocytosed). Bacteria can also coat itself in a plasma protein and then use this mechanism as a bridge to its reciprocal PLC receptors. For example, clumping factor A & B and fibronectin binding protein on S. aureus can both bind fibronectin and/or fibrinogen both of which are ligands for GPIIb/IIIa on the PLCs.

Bacterial protein (e.g., proteins that can directly interact with a surface receptor on the PLCs. A non-limiting example of the scavenging effect of PLCs is demonstrated by Toll Like Receptor (TLR) 1, 2, 4, 6, 8 and 9, type I integral membrane receptors, presented by the PLCs recognize common pathogen-associated molecular patterns found in foreign invaders. Some of the common PLC receptors directly or recognized by bacteria or bacterial proteins include, but are not limited to Glycoprotein Ibα, Glycoprotein IIbIIIa, FcγRIIA, gC1q-R/P32, Toll like receptor 2.

PLCs or derivatives thereof may also be used as scavengers to clear blood-derived toxins, including but limited to uremic toxins, toxins from pesticides, metal toxins, such as but not limited to lead, iron, cadmium. arsenic, manganese, nickel, chromium, or vanadium, toxins derived from snake bites, spider bites or other insect, reptile or animal bites. A common feature between clearance of autoantibodies, viruses, bacteria, blood related toxins or any unwanted elements in the body is the property of PLCs' receptors, ligands or antigens (endogenous to PLCs or exogenously expressed) to scavenge for these harmful elements, deliver these harmful elements primarily to the liver where they are cleared and degraded, often rapidly, thereby rendering them harmless. As an example, in the liver the PLCs or derivatives thereof selectively binds to liver-based receptors, such as, but not limited to, Ashwell-Morrell receptor (AMR). The receptor selectively binds to galactose or N-acetylgalactosamine residues of deasilylated glycoproteins and internalizes them thereby rapidly clearing from blood circulation glycoproteins bearing glycan ligands that include galactose and N-acetylgalactosamine, for example.

In some embodiments, PLCs or derivatives thereof often act as sponge, for example, through antigen-specific immunoadsorption. Here, the PLCs take the advantage of their large surface area and their membrane flexibility permits the PLCs to adsorb the autoantibodies, viruses, bacteria, blood related toxins or any unwanted elements in the body for their removal.

### Pharmaceutical Formulations of the PLCS or Derivatives Thereof

Therapeutic formulations of the PLCs or derivatives thereof used in accordance with the present disclosure are prepared for storage by mixing PLCs or derivatives thereof having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol(PEG), for example, a PEG chain having a molecular weight between 1,000-15,000 daltons, or between 2,000 and 10,000 daltons, or between 2,000 and 5,000 daltons. Other hydrophilic polymers which may be suitable include polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose.

Lyophilized formulations adapted for subcutaneous administration are also contemplated by the invention. Such lyophilized formulations may be reconstituted with a suitable diluent to an optimal concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a cytotoxic agent, cytokine or immunosuppressive agent. The effective amount of such other agents depends on the amount of PLCs or derivatives thereof present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi permeable matrices of solid hydrophobic polymers containing the PLCs or derivatives thereof, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyricacid. The formulations to be used for in-vivo administration must be sterile. This is readily accomplished by filtration through sterile filtration membranes.

Treatment with the PLCS or Derivatives Thereof

The composition comprising PLCs or derivatives thereof which binds to an autoimmune antibody or a viral protein will be formulated, dosed, and administered in a fashion consistent with good medical practice. Factors for consideration in this context include the particular disease or disorder being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the PLCs or derivatives thereof to be administered will be governed by such considerations.

As a general proposition, the therapeutically effective amount of PLCs or derivatives thereof administered parenterally per dose will be in the range of about 0.01 to 1000 mg/kg of patient body weight per day, with the typical initial range of PLCs or derivatives thereof used being in the range of about, 0.03-300 mg/kg or 0.05-100 mg/kg, or alternatively 0.1-75 mg/kg or 0.5-50 mg/kg.

Suitable dosages for PLCs or derivatives thereof are, for example, in the range from about 20 mg/kg to about 1000 mg/kg. For example, one may administer to the patient one or more doses of substantially less than 375 mg/kg of the PLCs or derivatives thereof e.g. where the dose is in the range from about 20 mg/kg to about 250 mg/kg, for example from about 50 m g/kg to about 200 mg/kg. For example, the initial dose may be in the range from about 0.1 mg/kg to about 100 mg/kg, or 10 mg/kg to about 250 mg/kg (e.g. doses of 0.3-60 mg/kg over a 2-h infusion including 4 weekly doses of 15 or 30 mg/kg) and the subsequent dose may be in the range from about 1 mg/kg to about 10 mg/kg. Administration may be in a single dose or may e.g. occur every 3 to 4 hours, 1-4 times a day, 1-4 times a week; 1-4 times a month, possibly 1-7 times a week, or possibly administration occurs once every 3 or 4 weeks. As will be appreciated by the person skilled in the art, the dose of the PLCs or EVs or derivatives thereof, according to the present disclosure, may depend on many factors and optimal doses can be determined by the skilled person via routine experimentation.

These suggested amounts of PLCs or derivatives thereof are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the PLCs or derivatives thereof is administered as close to the first sign, diagnosis, appearance, or occurrence of the disease or disorder as possible or during remissions of the disease or disorder.

The PLCs or derivatives thereof is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the PLCs or derivatives thereof may suitably be administered by pulse infusion, e.g., with declining doses of the PLCs or derivatives thereof. In some embodiments the dosing is given by injections, most preferably intravenous or subcutaneous injections, depending in part on whether the administration is brief or chronic.

One may administer other compounds, such as cytotoxic agents, immunosuppressive agents and/or cytokines with the PLCs or derivatives thereof herein. The combined administration includes co administration, using separate formulations or a single pharmaceutical formulation, and consecutive administration in either order, wherein preferably there is a time period while both (or all) active agents simultaneously exert their biological activities.

In some embodiments, a diagnostic method or method of screening is provided for toxic agents such as autoimmune autoantibodies, antigens, viral or bacterial protein, or any other biological or chemical toxins, comprising: (a) obtaining a sample from a subject in which the presence of one or more of these agents is suspected; (b) admixing with the patient sample a composition comprising PLCs or EVs, platelet variants or derivatives thereof that exogenously or endogenously express one or more receptors/ligands/antigens for a counterpart ligand/receptor or an antigen to which agents such as autoimmune antibodies, or viral entry receptor proteins interact with or bind to; and (c) determining the presence or absence of the autoimmune antibody, or the bacterial or viral particles or viral peptides or viral nucleic acids in the patient's sample. Labelling techniques such as radiolabel, a fluorophore, a chromophore, an imaging agent or a metal ion may be employed to assist in such diagnosis. Labeling techniques are well known to one of skill in the art.

Further details of the disclosure are illustrated by the following non-limiting Examples. These examples are put forth so as to provide those of ordinary skill in the art with a complete disclosure and description of how to make and use the compositions and methods of the disclosure, and are not intended to limit the scope of what the inventors regard as their disclosure.

### EXAMPLES

### Example 1: Exemplary Antibodies such as Anti-CD41/61 can be Quantified in Mouse Blood and Plasma

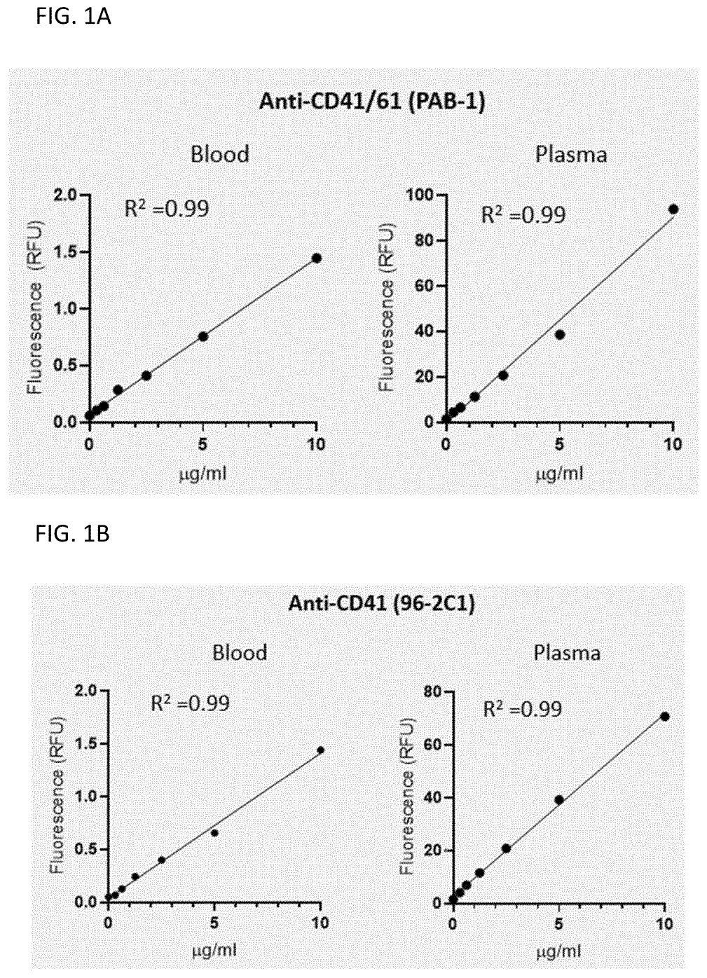

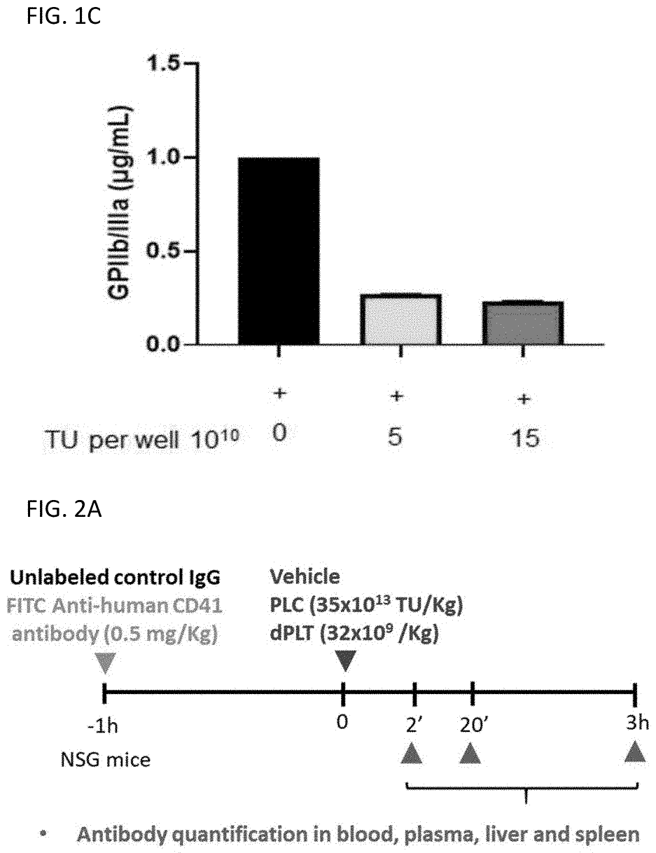

A study was designed to examine whether fluorescently labeled anti-CD41/61 can be quantified in mouse blood and plasma. FITC-conjugated mouse anti-human CD41/61 was added to 1 mL of mouse blood to achieve 10 mg/ml concentration. Serial dilutions (5, 2.5. 1.25, 0.62, 0.31, 0 mg/ml) were prepared by diluting the 10 mg/ml in mouse blood. Aliquots of mouse blood were transferred into wells of a 96 well plate and fluorescent signal (525 nm) was acquired by a plate reader. The remaining blood was then centrifuged at 20,000 g for 20 minutes to obtain plasma. Mouse plasma was then transferred into wells of a 96 well plate and fluorescent signal (525 nm) was acquired by a plate reader. The study results indicate that fluorescently labeled anti CD41/61 antibody can be measured in mouse blood and plasma (FIGS. 1A through 1C, as indicated below). The standard curve obtained can be used to calculate the antibody concentration in the blood of mice dosed with anti CD41/61 (PAB-1) (FIG. 1A) and anti CD41 (96-2C1) antibodies (FIG. 1B). FIG. 1C shows PLCs (PB101) binds fluorescently labeled Anti-CD41/61 antibodies (TU=PLCs/well).

### Example 2: PLCs Clear Exemplary Anti-CD41 Antibody (96-2C1) and Anti CD41/61 Antibody (PAB-1) In-Vivo and the Clearance Predominately Occurs in Liver

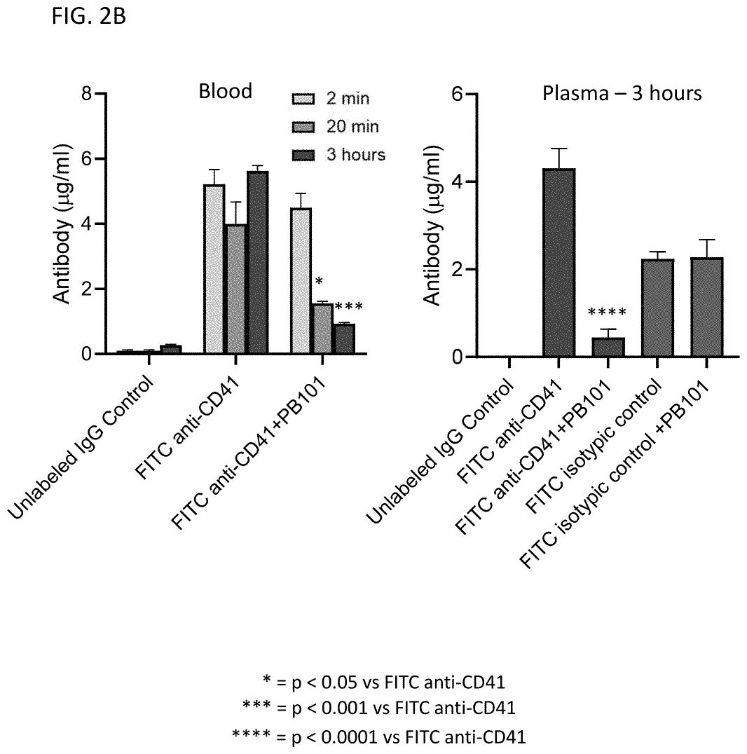

To determine if PLCs can clear anti-CD41 antibody (96-2C1) and anti CD41/61 antibody (PAB1) in-vivo, NSG immunodeficient mice were dosed with a fluorescently labeled mouse anti-human-CD41 (96-2C1) or CD41/61 FITC antibody (PAB-1) at 0.5 mg/kg. FIG. 2A is an illustration of how the experiments were performed for the quantification of CD41 antibody in blood plasma, liver, and spleen. Antibody (CD41) was injected 1 hour prior to PLC or donor platelet (PLT) treatment (referenced to as −1; FIG. 2A). Antibody concentration was quantified at 2 min, 20 min and 3 hours in blood and only after 3 hours in spleen and liver after injecting with the PLCs or PLTs. These antibodies do not recognize or cause clearance of mouse platelets.

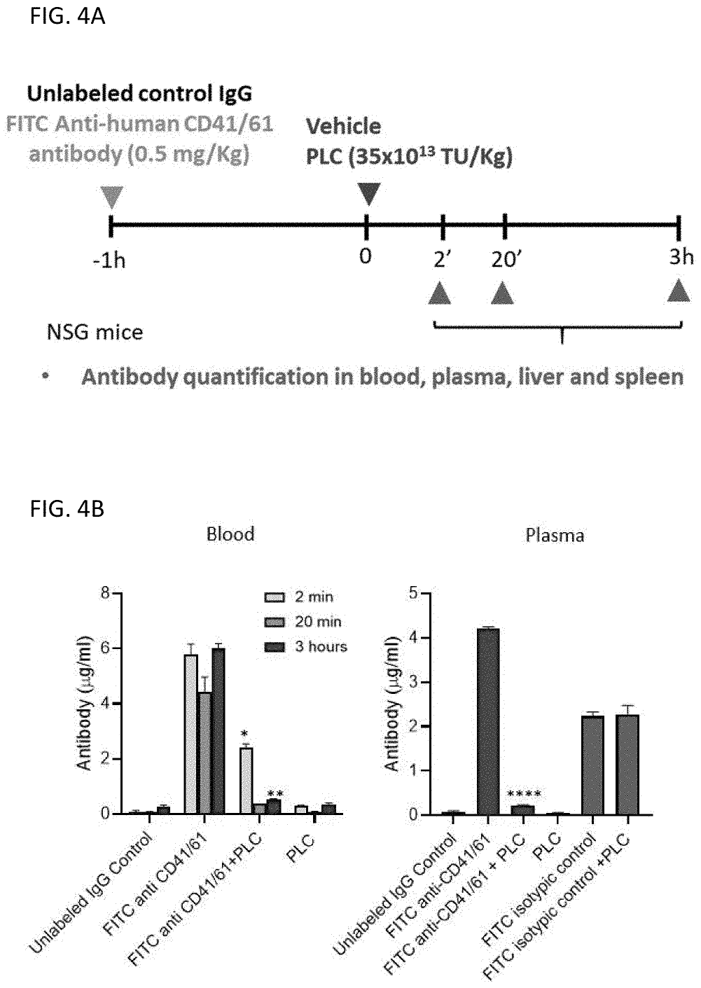

One hour after anti-CD41 (FIG. 2B) or anti-CD41/61 antibody (FIG. 4B) administration, a PLCs (PB101) dose of 32×10\hat{ }9 PLCs/kg (also referred to as Platelet Equivalent Unit (PEU)/kg) or Plasma-Lyte A, a buffer control, were administered to the mice (n=3/group). Blood was collected via tail vein at 2 mins and 20 mins and via cardiac puncture at 3 hours. Antibody quantification via fluorescence in blood was assessed at 2 mins, 20 mins, and 3 hours after PLCs were administered using a plate reader and fluorescence in plasma was assessed at 3 hours. In addition, a comparison of antibody fluorescence between the liver and the spleen was assessed at 3 hours by flow cytometry.

The data in FIGS. 2B, 3B, 4B, and 5B indicate that a marked decrease in antibody fluorescence in blood was observed at about 2 mins with a greater decrease at about 20 mins and effectively fully cleared at about 3 hours for PLCs treated mice. Similarly, a significant decrease in antibody fluorescence in plasma was observed at 3 hours for PLCs treated mice.

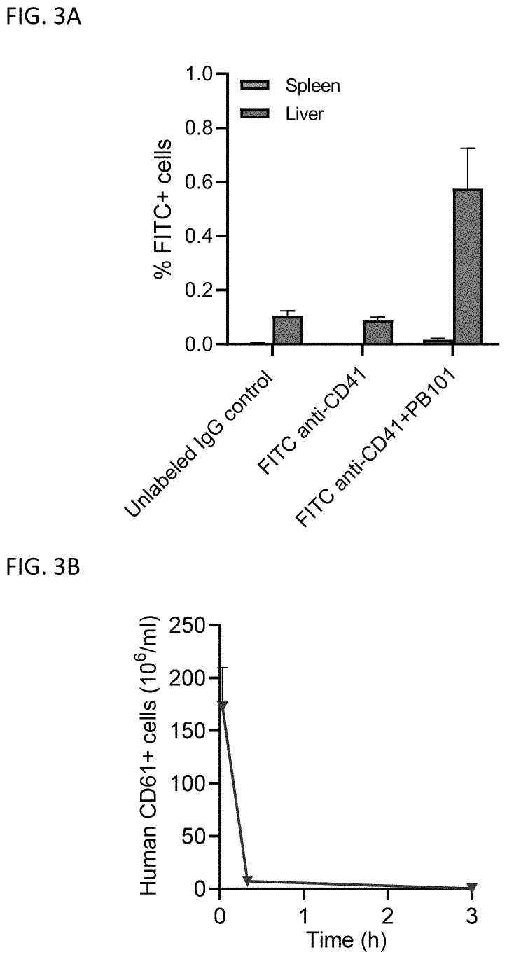

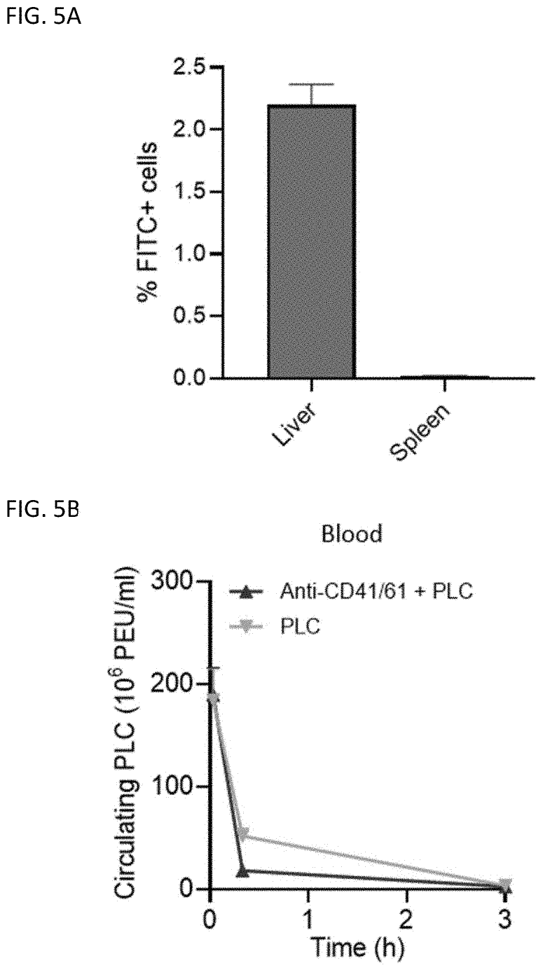

To determine if PLCs can clear anti-CD41 antibody (96-2C1) and anti CD41/61 antibody (PAB-1) in-vivo in liver a protocol similar to that described in FIG. 2A was used except that CD41/CD61 antibodies were used (FIG. 4A) and that the fluorescence was quantified in homogenized livers and spleens at the end of the study. Results in FIGS. 3A and 5A demonstrate that PLC-dependent anti-CD41 antibody (96-2C1) and anti CD41/61 antibody (PAB1) clearance substantially occurs in the liver.

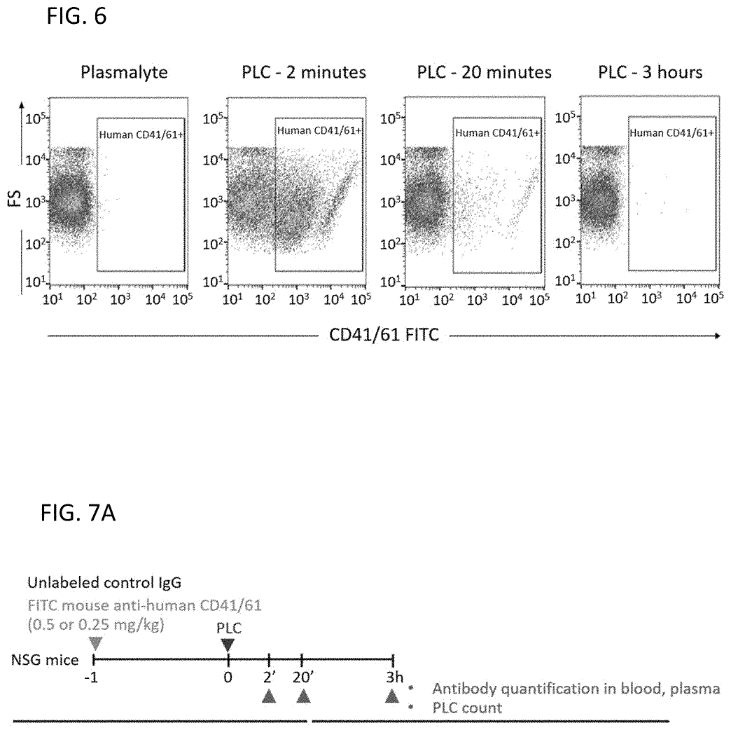

Further, to study the time-dependent binding of PAB-1 to PLCs was studied in-vivo. Experimental protocol for this study was similar to that shown in FIG. 4A. Results shown in FIG. 6 demonstrate that optimum binding of PLCs to anti-CD41/61 antibody (PAB-1) in-vivo occurs between 2 and 20 minutes. The data indicate that PLCs bind to the anti-CD41/61 antibodies as determined by flow cytometry.

### Example 3: Clearance of Exemplary ITP Antibodies is Titratable with PLC Dose

The in-vivo efficacy of various PLCs doses to bind and clear anti-CD41/61 antibodies in NSG mice was evaluated. The mice were dosed with a mouse anti-human-CD41/61 FITC antibody (PAB-1) at 0.5 mg/kg, or 0.25 mg/kg.

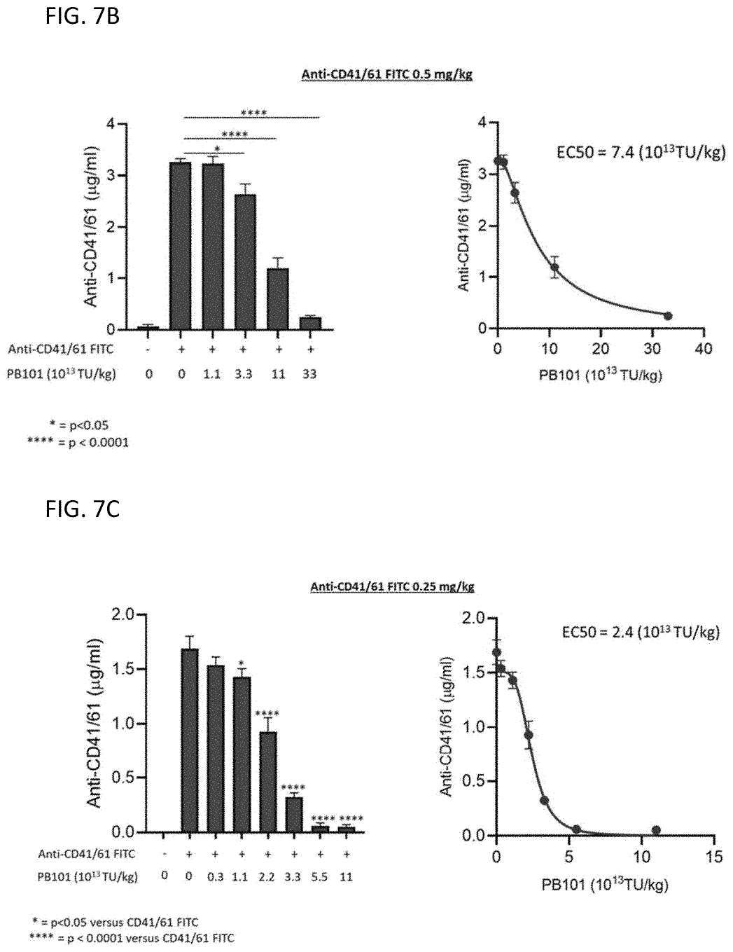

One hour after anti-CD41/61 antibody administration, various doses of PLCS or Plasma-Lyte A, a buffer control, were administered to the mice (n=6/group). Blood was collected via tail vein at 2 mins and 20 mins and via cardiac puncture at 3 hours. Antibody quantification via fluorescence in blood was assessed at 2 mins, 20 mins, and 3 hours and fluorescence in plasma was assessed at 3 hours (FIG. 7A).

The data show a dose dependent decrease in antibody fluorescence with an EC₅₀ of 7.4 and 2.4×10¹³ Platelet Equivalent Unit (PEU)/kg, respectively for the high dose of antibody (0.5 mg/kg) (FIG. 7B) and for the low dose (0.25 mg/kg) (FIG. 7C). FIGS. 7B and 7C show PLCs are titratable and a reduction in EC50 is achieved with anti-CD41/61 antibody dosing comparable to levels in ITP patients.

### Example 4: PLCs-Mediated CD41/61 Antibody Clearance Through the Liver Results in Upregulation of Thrombopoietin (TPO) Production In-Vivo.

A study was performed in order to evaluate if PLCs-mediated. CD41/61 antibody clearance through the liver results in upregulation of thrombopoietin (TPO) production in-vivo. Immunocompromised NOD scid gamma (NSG) mice were injected intravenously (i.v.) with 0.5 mg/kg of mouse anti-human CD41/61 (clone PAB-1). After 1 hour, the mice were dosed i.v. with plasmalyte (vehicle), donor platelets (30×10⁹ /kg) or PLCS (33×10¹³ TU/kg.). Mouse Blood was collected in EDTA tubes by cardiac puncture 24 hours after PLCS injection. The blood was centrifuged at 20,000 g for 20 minutes to obtain plasma. Thrombopoietin levels in plasma were quantified by ELISA,

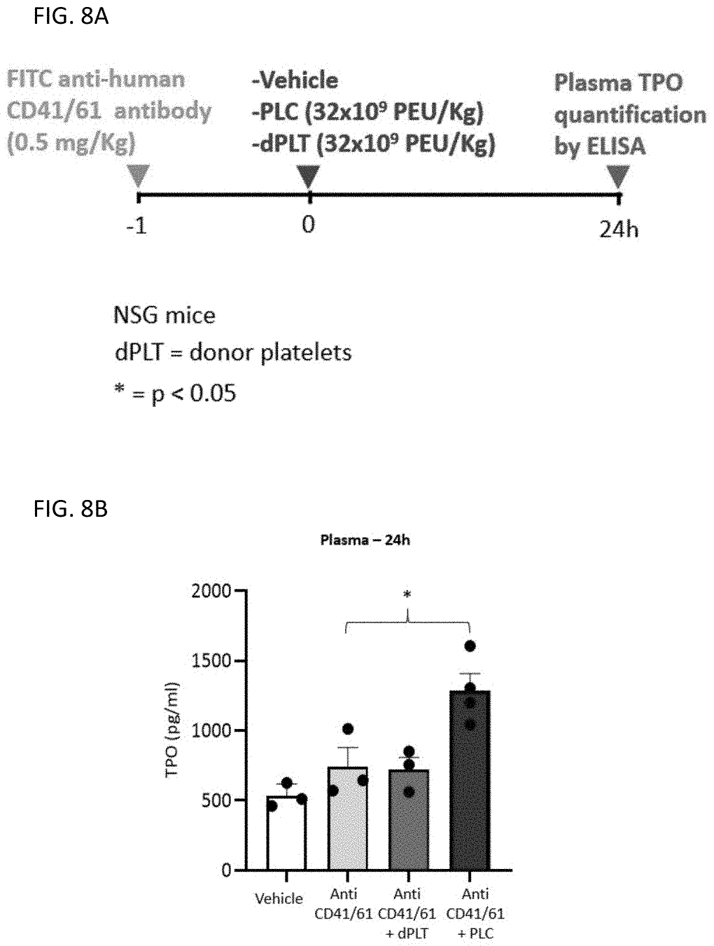

Experimental design in FIG. 8A is essentially similar to that described in FIG. 4A. In brief, Antibodies (CD41/61) were injected 1 hour prior to PLC or PLT treatment (referenced to in FIG. 8A as (−1)). After the PLC/PLT treatment, plasma TPO quantification was performed 24 hours after. The data in FIG. 8B show that anti-CD41/61 clearance mediated by PLCs results in a significant upregulation of plasma TPO in-vivo. On the contrary, no significant changes in plasma TPO levels were observed in plasma of mice treated with anti CD41/61 antibody or with anti CD41/61 antibody and donor platelets.

### Example 5: PLCs Express High Levels of Receptors, such as Glycoproteins IIb/IIIa Antigens, that Bind and Clear the Pathogenic Autoantibodies

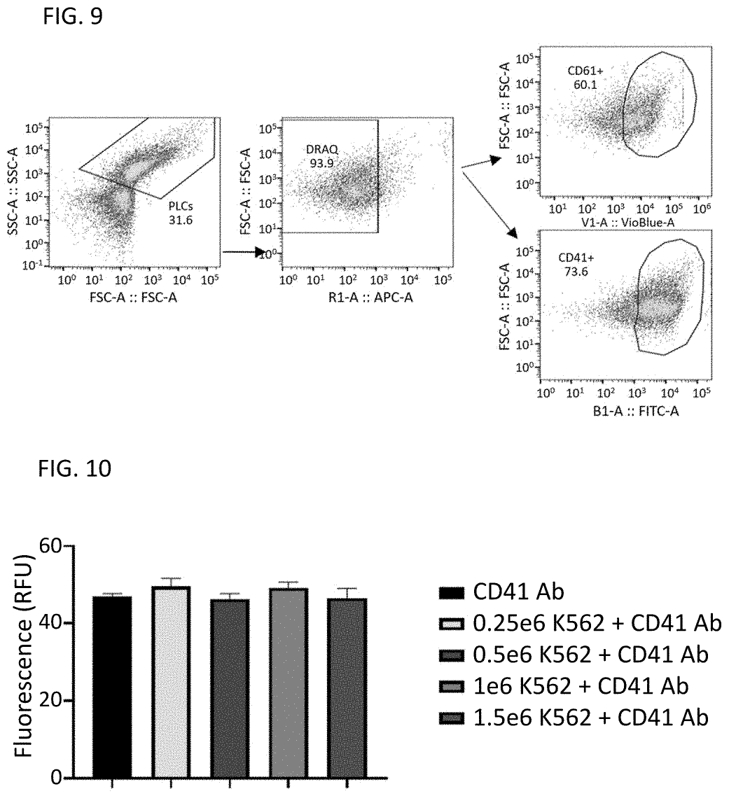

To assess the expression of IIb/IIIa on PLCs, cells are stained with antibodies against CD41, CD61, and the nuclear dye DRAQ5 by flow cytometry using Miltenyi MACSQuant. Cells were diluted in Plasmalyte solution (Baxter) to approximately 10\hat{ }6 and marked according to the manufacturer's instructions (Miltenyi). For surface expression analysis, cells are initially negative selected for DRAQ5 and placed within a size gate of approximately 1 μm to 10 μm. Following the parent gates, cells are then determined for expression of CD41 or CD61 independently (FIG. 9). The percent positive is determined by the appropriate IgG staining gate and selected on the shift in fluorescent signal. Final analysis of scatter plots were completed using FlowJo.

### Example 6: Specificity of the Removal of Anti-Glycoprotein Antibodies (Negative Control)

In order to establish the specificity of binding, K562, a human erythroleukemic cell line, known to lack expression of IIb or IIIa on the surface was purchased from ATCC. 1 μg/ml of FITC-labeled anti-CD41/61 antibody was incubated with increasing cell counts in a Plasmalyte solution. Following a 30-minute incubation at room temperature, the cells were removed from the antibody solution by centrifugation. The remaining supernatant was measured for the presence of the FITC-labeled anti-CD41/61. Because K562 lacks the expression of IIb or IIIa, anti-CD41/61 antibody showed no binding and removal of antibody (FIG. 10).

### Example 7: Increasing PLCs Leads to Enhanced Reduction of Autoantibodies

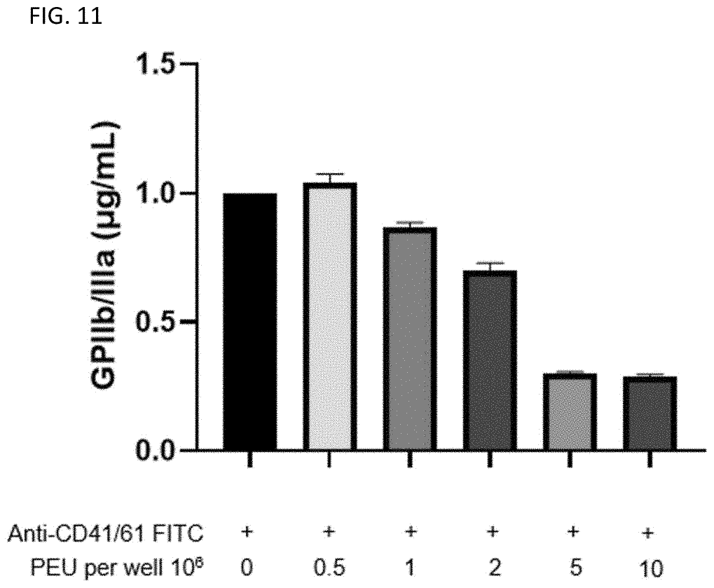

In-vitro studies were designed to test whether the PLCs can bind and remove anti-glycoprotein IIb/IIIa antibodies. Here, the binding of PLCs to FITC-labeled anti-CD41/61 antibodies (American Research Products) was evaluated in-vitro for potency. 1 μg/mL of antibody was incubated in the presence of PLCs for 30 mins in a Plasmalyte solution. The PLCs were removed from solution using a centrifugal filter. The total remaining fluorescence was measured by fluorescent plate reader (485 nm) and compared to the antibody alone. The reductions in antibody fluorescence indicated the amount of antibody that was bound and removed by the PLCs from the Plasmalyte solution. Data is graphed representing the amount of antibody removed per PLC concentration (FIG. 11).

### Example 8: Exemplary Identification of Diverse Sets of Surface Antigens on the PLCs that are Capable of Binding and Clearing the Pathogenic Autoantibodies

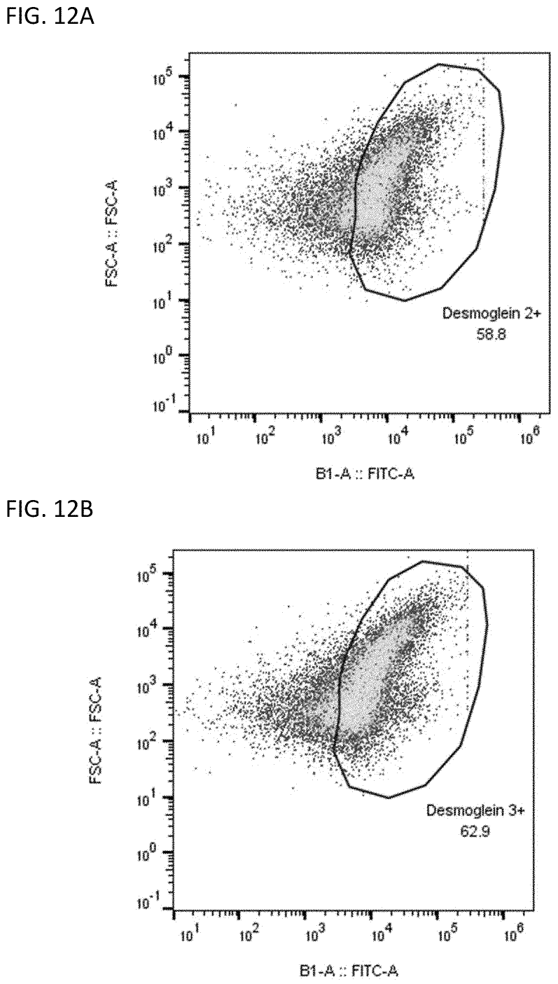

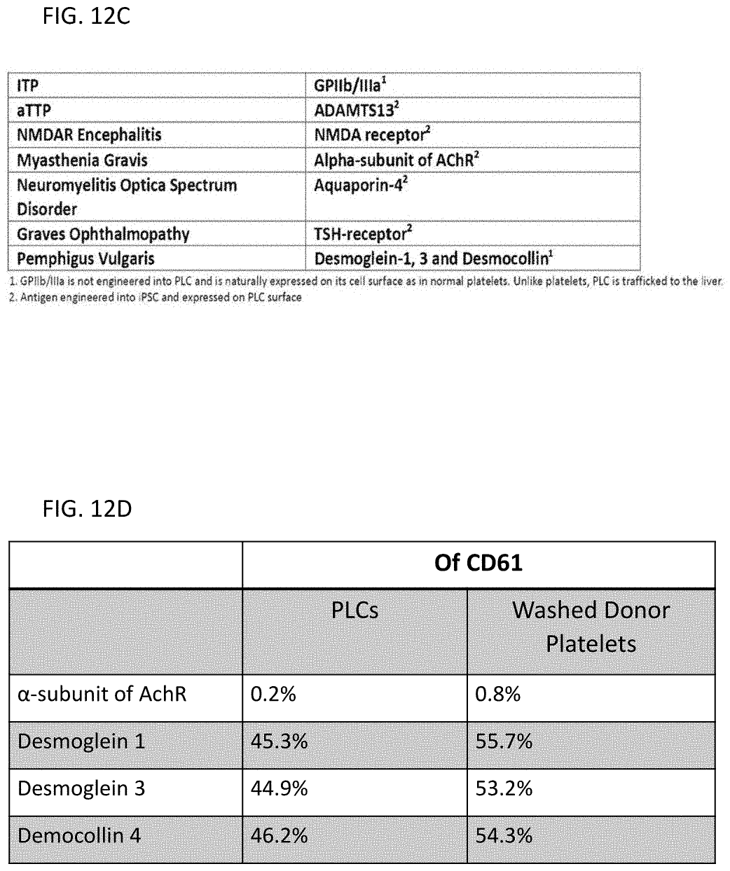

To assess the expression of diverse sets of surface antigens, antibodies against known surface antigens involved in autoimmune diseases, such as but not limited to desmoglein 2, desmoglein 3 were purchased from commercial vendors. Cells were identified by CD61 expression and free of nuclear content by flow cytometry using a Miltenyi MAC SQuant. Cells were diluted in Plasmalyte-A and stained according to the antibody manufacturer's instruction. For expression analysis, cells were initially negative selected for DRAQ5 and CD61 positive events. Following the parent gates, the expression of autoantibody surface antigens, desmoglein 2, desmoglein 3, were determined. FIGS. 12A and 12B provide examples of removal of autoantibodies by the PLCs, as exemplified by desmoglein 2 and desmoglein 3. In a manner similar to that shown in FIGS. 12A and 12B, binding of autoantibodies to diverse sets of surface antigens, such as, Rh Factors, Acetylcholine receptor alpha subunit, aquaporin 4, desmocollin 4, thyroid stimulating hormone receptor, Factor H, ADAMT13, NMDAR1, myeloperoxidase, collagen IV, proteinase, bovine ganglioside, contactin 1, whether endogenously or exogenously expressed in the PLCs, can be demonstrated. FIGS. 12C-12D illustrate that PLCs' mechanism of action lends itself to expansion of the platform to diverse antibody mediated autoimmune diseases. FIG. 12C summarizes some of the autoimmune disease receptors that are (1) endogenously expressed in the PLCs or (2) can be exogenously expressed in the PLCs. FIG. 12D is an exemplification, which shows concentration of some of the autoimmune disease receptors in the PLCs vs donor platelets.

### Example 9: Clearance of Exemplary Antibodies: Diverse Antigens Present on PLC Surface that can Bind and Clear Autoimmune Disease-Causing Circulating Antibodies

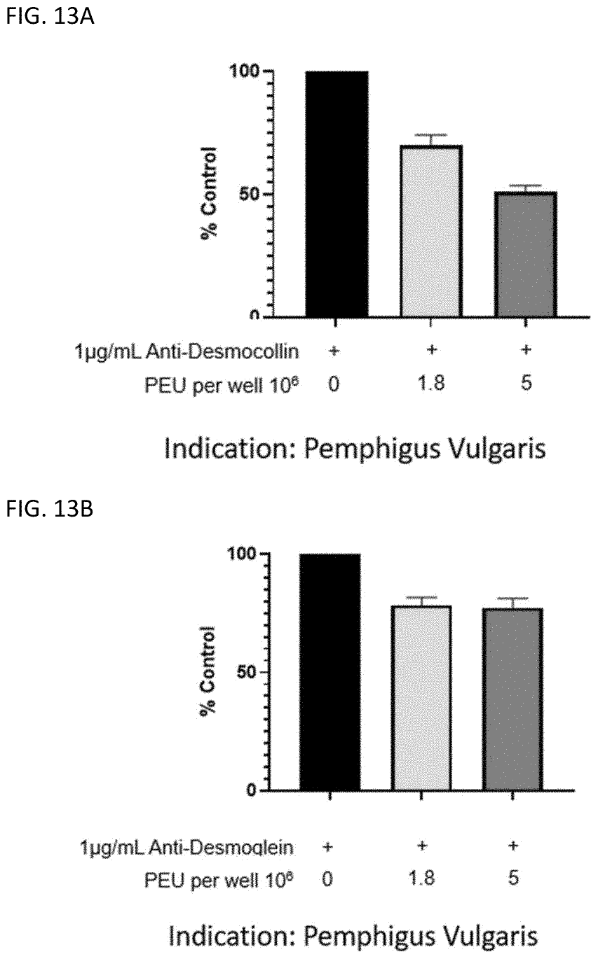

Further, to support that PLCs clear autoimmune disease-causing circulating antibodies, the binding of PLCs to FITC-conjugated antibodies was evaluated for in-vitro potency of antibody removal as show in FIGS. 13A-13B. In FIGS. 13A-13B, 1 μg/mL of FITC-labeled antibody of anti-desmocollin (FIG. 13A) or 1 μg/mL of FITC-labeled antibody of desmoglein (FIG. 13B) was incubated in the presence of two concentrations of PLCs for 30 minutes in a Plasmalyte solution. The removal of PLCs from the buffer solution was completed using centrifugation and a centrifugal filter.

As shown in FIG. 13A (desmocollin) and FIG. 13B (desmoglein), PLCs were capable of binding and remove autoimmune disease-causing circulating antibodies (exemplified here for the autoimmune disease pemphigus vulgaris. In a manner similar to that shown in FIGS. 13A and 13B, binding of autoantibodies to diverse sets of surface antigens, such as, Rh Factors, Acetylcholine receptor alpha subunit, aquaporin 4, desmocollin 4, thyroid stimulating hormone receptor, Factor H, ADAMT13, NMDAR1, myeloperoxidase, collagen IV, proteinase, bovine ganglioside, contactin 1, TSH-receptor, can be shown for their removal of autoantibodies by the PLCs or ePLCs expressing the surface antigens.

FIG. 14A is an example of a lentiviral vector that can be used to exogenously express into the PLCs diverse set of antigens, such as, α-subunit of AChR, Aquaporin-4, ADAMTS13, NMDAR, TSH-receptor, Phospholipase A2R etc., that can be presented by the genetically engineered PLCs to bind to and clear circulating autoantibodies.

### Example 10: An Example of a Lentiviral Vector with the ORF so Designed for Exogenous Expression of AI Receptors, Ligand or Antigens in the PLCs or EVs

FIG. 14A is a specific example of one of the lentiviral vectors with the AcHR alpha subunit ORF so designed for myasthenia gravis. FIG. 14B(i) through 14B(v) shows that the AcHR alpha subunit ORF can be routinely replaced with a gene of interest as exemplified with the genes for different domains of (i) Myasthenia Gravis (FIG. 14B(i)), or with (ii) a neuromyelitis Optica Spectrum disease (FIG. 14B(ii)), or with Thrombotic Thrombocytopenic Purpura (FIG. 14B(iii)) or anti-NMDA receptor encephalitis (FIG. 14B(iv)) or Membranous Nephropathy (FIG. 14B(v)).

### Example 11: Receptors Generating Autoantibodies can be Exogenous Receptors as Exemplified by Expression of Acetylcholine Receptor (AchR) Receptors

Myasthenia gravis (MG) is caused by an abnormal immune reaction (antibody-mediated autoimmune response) in which the body's immune defenses (i.e., antibodies) inappropriately attack certain receptors in muscles that receive nerve impulses. More specifically, MG is caused by autoantibody production against proteins of the neuromuscular junction (NMJ). Autoantibodies appear specific to the alpha chain of the acetylcholine receptor, a 5-subunit receptor expressed at the NMJ).

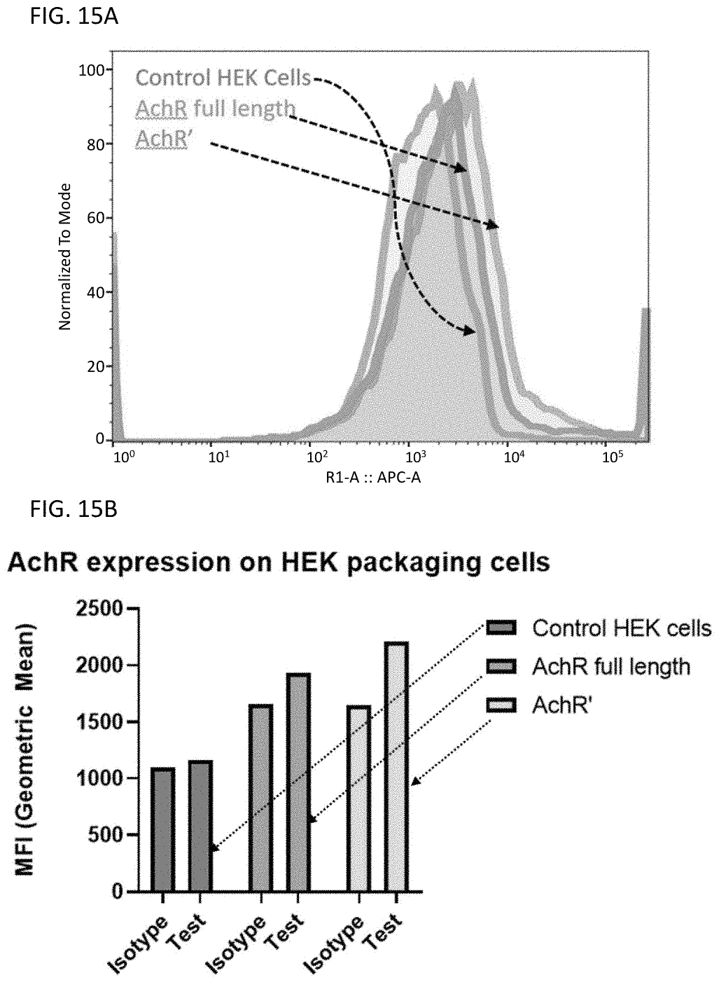

In order to determine the expression level of the expression constructs that exogenously encode for the full length AchR alpha 1 (black arrow) and the truncated version referred to as AchR′ (broken arrow), a flow cytometry was performed using a AF647 conjugated antibody that recognizes endogenous AchR alpha 1. HEK293 cells that had been transfected with these expression constructs were used in order to produce lentivirus for future experiments. As shown in FIGS. 15A and 15B, there was an increase in antibody binding to HEK293 cells transfected with AchR alpha 1 (short arrow) and AchR′ (long arrow) as compared to the control HEK293 cells (curved arrow). These results exemplify that that the expression constructs present epitopes, for example, specific to the AchR alphal protein that can be detected by commercially available antibodies directed against the endogenous protein.

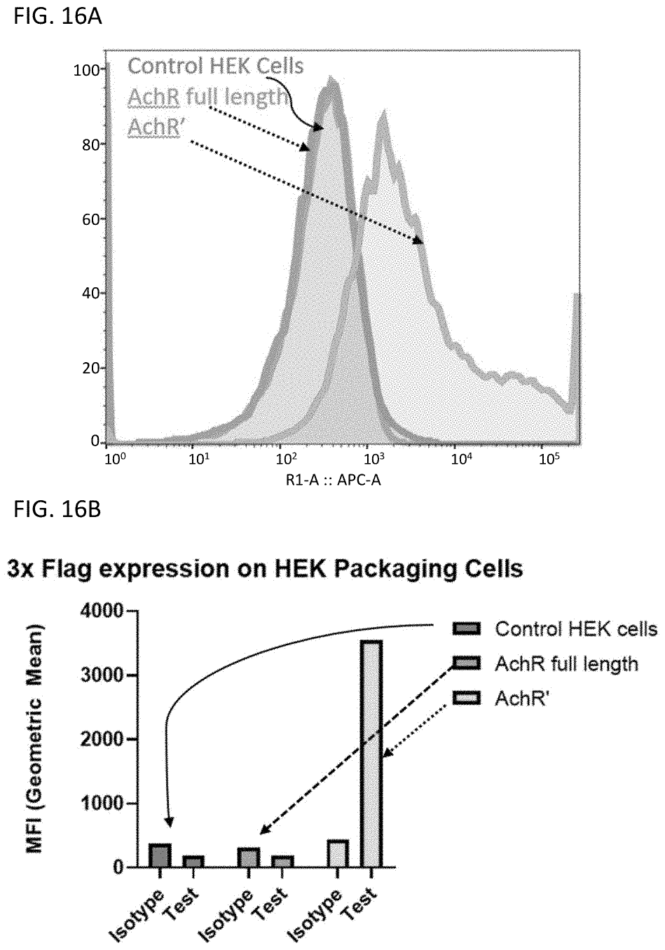

In order to determine the expression level of the 3X FLAG affinity tag on the N-terminus of the AchR′ peptide (broken arrow), a flow cytometry was performed using an allophycocyanin (APC) conjugated antibody that recognizes proteins with the 3X FLAG tag (FIG. 16A). HEK293 cells that had been transfected with these expression constructs were used in order to produce lentivirus for future experiments. As shown in FIG. 16B, there was an increase in antibody binding to HEK293 cells transfected with the AchR′ peptide (short arrow) and no observable antibody binding to control HEK cells (curved arrow) and cells transfected with the full length AchR alpha 1 that lacks a 3X FLAG affinity tag (long arrow). These results demonstrate that the AchR′ peptide successfully shuttles to the cell membrane and can be detected by antibodies directed against the 3X FLAG affinity tag. Likewise, engineered PLCs (ePLCs) can be generated expressing, for example, the full length AchR or extracellular domain of the AchR (AchR′) peptide. Such ePLCs can be used as baits to bind to autoimmune antibodies, as taught throughout this invention, to remove or eliminate such autoantibodies that are causative agents for autoimmune diseases, such as for example, Myasthenia gravis.

### Example 12: IL-12 Protein Expression is Upregulated in Engineered EVs Derived from PLC-Producing Progenitor Cells Exogenously Expressing IL-12

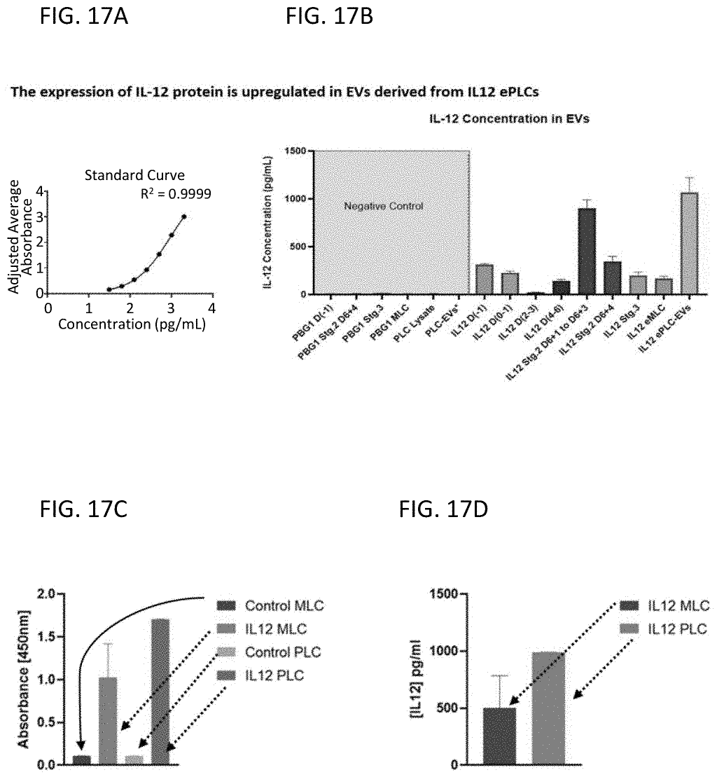

To determine whether protein of interest can be loaded into the PLC-EVs through the approach of molecular engineering, IL-12 is selected as a proof-of-concept protein. Engineered iPSCs (eiPSCs) expressing IL-12 were developed, followed by differentiation, and underwent Bioreactor run to generate IL-12 expressing engineered PLCs (IL-12 ePLCs). The EVs were isolated from the spent media during differentiation and the supernatant of the bioreactor run. Proteins were extracted from these EVs using RIPA (radioimmunoprecipitation assay buffer) buffer supplemented with protease inhibitor cocktails, then subjected to BCA assay for concentration determination. The protein amount was normalized to 20 μg for each sample, and the IL-12 protein concentration was measured using human IL-12 p70 ELISA kit (R&D Systems) as per the instructions. The result (FIGS. 17A and 17B) indicated that the IL-12 protein expression was low or below detection limit in the EVs that were derived from PBG1 control cells (iPSCs, MLCs, and PLCs). On the other hand, the IL-12 expression was significantly elevated in the EVs (engineered EVs) that were isolated during the IL-12 eiPSC differentiation (FIG. 17B). Furthermore, the IL-12 expression in the IL-12 eMLC and IL-12 ePLC-EVs were also elevated, and the concentration was 169 and 1066 pg/mL, respectively (FIG. 17A). This study demonstrated that by molecular engineering of iPSCs followed by differentiation and bioreactor run, the protein of interest (i.e., IL-12) can be efficiently loaded into the ePLC-EVs.

### Example 13: IL-12 Protein Levels and Expression in Genetically Engineered PLCs

In FIG. 17B, IL-12 protein levels were quantified by ELISA in PBG1 (i.e., iPSCs) untransduced control cells, the antibiotic-selected IL-12 population cells and individual single cell iPSC clones grown from the IL-12 transduced population. Increased IL-12 protein was observed in 2 clones and one of these clones was selected for further development. Quantitation of IL-12 protein in control (untransduced) PBG1 differentiated cells and cells differentiated from the IL-12 expressing clone was performed by ELISA on cell lysates. Increased IL-12 protein was observed in both MLC and PLC compared to control cells (FIG. 17B). Quantitation of exogenously expressed IL-12 protein based on the assay standard curve indicates protein quantities in the picogram range for MLC and near nanogram range for PLC (FIG. 17C and 17D).

It is understood from the examples disclosed herein that it will be routine practice for one of skilled in the art to carry out similar studies. For example, IL-12 can be substituted with other genes of interest (e.g., viral, bacterial or of biological toxins) to produce genetically engineered PLCs (ePLCs), the expressions of which can be characterized in the same manner as described in FIGS. 17A through 17D and throughout the application.

### Example 14. PB101 Clearance of Anti-Platelet Antibodies is Likely Mediated via AMR and Induces Production of TPO

Treatment of thrombocytopenic ITP patients with PLCs is expected to result in the clearance of pathogenic autoantibodies concomitant with induction of TPO and new platelet production. The two events are expected to be synergistic and lead to a clear clinical benefit in ITP patients. PLC administration in animals increases the physiological functions of endogenous platelets by competing and binding to autoantibodies that otherwise target autoantigenic endogenous cell. In this study, a mouse model for autoimmune disease will be subjected to treatment with PLCs or derivatives thereof and monitored for amelioration or elimination of the AI disease induced by the PLCs or derivatives thereof. For example, a mouse model that produces anti mouse CD61 antibodies, which cause thrombocytopenia, will be injected with PLCs. It is expected that PLC-induced clearing of the circulating autoantibodies will rescue thrombocytopenia in mice.

From the foregoing description, it will be apparent that variations and modifications may be made to the embodiments of the present disclosure to adopt it to various usages and conditions. Such embodiments are also within the scope of the following claims. The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating or ameliorating an autoimmune disease or symptom thereof in a patient in need of such treatment, the method comprising administering to the patient a therapeutically effective amount of platelet-like-cells (PLCs) or derivatives thereof.

2. The method of claim 1 wherein the derivative comprises genetically engineered PLCs derived from a PLC-producing progenitor cell where the PLCs are transformed to express at least one receptor, ligand or an antigen commonly shared with an endogenous autogenic receptor, ligand or an antigen.

3. (canceled)

4. The method of claim 1 wherein the disease is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Neuromyelitis Optica Spectrum Disorder, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, Membranous Nephropathy, IgG4-related sclerosing disease, Immune thrombocytopenic purpura (ITP), acquired thrombotic thrombocytopenic purpura (aTTP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neuromyelitis Optica Spectrum Disorder, Neutropenia, NMDAR encephalitis, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus vulgaris, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), acquired thrombotic thrombocytopenic purpura (aTTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and a combination thereof.

5. The method of claim 1 wherein the disease is selected from one or more of Immune thrombocytopenic purpura, Myasthenia gravis, Neuromyelitis Optica Spectrum Disorder, anti-N-methyl-D-aspartate receptor (NMDAR) encephalitis, Myasthenia Gravis, Graves Ophthalmology, Acquired thrombotic thrombocytopenic purpura (aTTP), or Pemphigus vulgaris and a combination thereof.

6. The method of claim 1 wherein the disease comprises induction of an inflammatory response.

7. The method of claim 6 wherein the inflammatory response is selected from the group consisting of psoriasis, dermatitis, systemic scleroderma, sclerosis, respiratory distress syndrome, dermatitis, meningitis, encephalitis, uveitis, colitis, glomerulonephritis, eczema, asthma, conditions involving infiltration of T cells and chronic inflammatory responses, atherosclerosis, leukocyte adhesion deficiency, rheumatoid arthritis, systemic lupus erythematosus (SLE), Type I diabetes mellitus, insulin dependent diabetes mellitis, multiple sclerosis, Reynaud's syndrome, autoimmune thyroiditis, allergic encephalomyelitis, juvenile onset diabetes, and immune responses associated with acute and delayed hypersensitivity mediated by cytokines and T-lymphocytes in tuberculosis, sarcoidosis, polymyositis, granulomatosis, vasculitis, pernicious anemia (Addison's disease), diseases involving leukocyte diapedesis, central nervous system (CNS) inflammatory disorder, multiple organ injury syndrome, hemolytic anemia, cryoglobinemia, Coombs positive anemia, myasthenia gravis, antigen-antibody complex mediated diseases, anti-glomerular basement membrane disease, antiphospholipid syndrome, allergic neuritis, Graves' disease, Lambert-Eaton myasthenic syndrome, autoimmune polyendocrinopathies, Reiter's disease, stiff-man syndrome, Behcet disease, giant cell arteritis, immune complex nephritis, IgM polyneuropathies, and autoimmune thrombocytopenia or combination thereof.

8. A method of treating or ameliorating an autoimmune diseases or symptom thereof in a patient in need of such treatment, the method comprising administering to the patient a therapeutically effective amount of an admixture comprising at least PLCs or derivatives thereof.

9. The method of claim 8 wherein the admixture further comprises PLC-derived extracellular vesicles (EV).

10. The method of claim 8 wherein the extracellular vesicles (EV) comprise microvesicles or exosomes or a combination thereof bioengineered or otherwise.

11. The method of claim 8 wherein the disease is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Neuromyelitis Optica Spectrum Disorder, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, Membranous Nephropathy, IgG4-related sclerosing disease, Immune thrombocytopenic purpura (ITP), acquired thrombotic thrombocytopenic purpura (aTTP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neuromyelitis Optica Spectrum Disorder, Neutropenia, Ocular cicatricial pemphigoid, NMDAR encephalitis, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), acquired thrombotic thrombocytopenic purpura (aTTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and a combination thereof.

12. The method of claim 1 wherein the disease is selected from one or more of Immune thrombocytopenic purpura, Myasthenia gravis, Neuromyelitis Optica Spectrum Disorder, anti-N-methyl-D-aspartate receptor (NMDAR) encephalitis, Myasthenia Gravis, Graves Ophthalmology, Acquired thrombotic thrombocytopenic purpura (aTTP) or Pemphigus vulgaris and a combination thereof.

13. A method of treating or ameliorating autoimmune diseases in a subject comprising administering to the subject PLCs or derivatives thereof comprising at least one receptor, an antigen or a ligand that is commonly shared with an autoantigenic endogenous receptor, an antigen or a ligand.

14. The method of claim 13, wherein the autoantigenic endogenous receptor, ligand or antigen is expressed in a cell in-vivo.

15. The method of claim 13 wherein an autoantigen on the autoantigenic endogenous receptor or ligand induces a humoral response generating autoantibodies.

16. The method of claim 13 wherein the at least one common receptor, ligand or antigen on the PLCs or derivatives thereof share the same autoantigen and specifically bind to the autoantibodies.

17. The method of claim 13 wherein the PLCs couple to autoantibodies selected from one or more of antinuclear antibodies, anti-transglutaminase antibodies, Anti-ganglioside antibodies, Anti-actin antibodies, anti-cyclic citrullinated peptide (CCP) antibodies, Liver kidney microsomal type 1 antibody, anti-thrombin antibodies, Antiphospholipid antibodies, Anti-neutrophil cytoplasmic antibodies, Rheumatoid factor (RF), anti-smooth muscle antibodies, anti-mitochondrial antibodies, anti-signal recognition particle (SRP) antibodies, anti-nicotinic acetylcholine receptor antibodies, or anti-muscle specific kinase antibodies.

18. The method of claim 13 wherein the disease is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Neuromyelitis Optica Spectrum Disorder, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, Membranous Nephropathy, IgG4-related sclerosing disease, Immune thrombocytopenic purpura (ITP), acquired thrombotic thrombocytopenic purpura (aTTP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neuromyelitis Optica Spectrum Disorder, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), acquired thrombotic thrombocytopenic purpura (aTTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and a combination thereof.

19. The method of claim 1 wherein the disease is selected from one or more of Immune thrombocytopenic purpura, Myasthenia gravis, Neuromyelitis Optica Spectrum Disorder, anti-N-methyl-D-aspartate receptor (NMDAR) encephalitis, Myasthenia Gravis, Graves Ophthalmology, Acquired thrombotic thrombocytopenic purpura (aTTP), or Pemphigus vulgaris and a combination thereof.

20. (canceled)

21. The method of claim 13, wherein the PLCs or derivatives thereof when administered are substantially devoid of generating an immune response.

22. The method of claim 13 further comprising administering to the subject an effective amount of a second therapeutic agent.

23. The method of claim 22 wherein the therapeutic agent is selected from one or more of antibodies or drugs.

24. A genetically engineered PLC or a derivative derived from a PLC-producing progenitor cell wherein the PLC is transformed to express at least one receptor, ligand or an antigen commonly shared with an endogenous autogenic receptor, ligand or an antigen and the endogenous autogenic receptor, ligand or the antigen couples to an autoimmune antibody in a patient.

25. The genetically engineered PLC or a derivative derived from a PLC-producing progenitor cell of claim 24 wherein the autoimmune antibody is generated from an autoimmune disease selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Neuromyelitis Optica Spectrum Disorder, Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, Membranous Nephropathy, IgG4-related sclerosing disease, Immune thrombocytopenic purpura (ITP), acquired thrombotic thrombocytopenic purpura (aTTP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neuromyelitis Optica Spectrum Disorder, Neutropenia, NMDAR encephalitis, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), acquired thrombotic thrombocytopenic purpura (aTTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and a combination thereof.

26. The genetically engineered PLC or a derivative derived from a PLC-producing progenitor cell of claim 24, wherein the autoimmune antibody is generated from an autoimmune disease selected from the group consisting of Myasthenia Gravis, Neuromyelitis Optica Spectrum Disorder, aTTP, NMDR Encephalitis, Graves Ophthalmology, and Pemphigus Vulgaris.

27. The genetically engineered PLC or a derivative derived from a PLC-producing progenitor cell of claim 24, wherein the PLCs or derivatives thereof are further transformed to express at least one receptor, ligand or an antigen from an X% to Y%, where Y% is greater than X% and the X% is an original concentration of the at least one receptor, ligand or an antigen on the PLC.

28. A method of treating a subject suffering from an autoimmune disease or a disorder by treating the subject with a therapeutic amount of the genetically engineered PLCs or the derivatives thereof of claim 24.

29. A pharmaceutical composition comprising the genetically engineered PLCs or their derivative of claim 24.

30. A method of treating or ameliorating a viral disease or a viral disease-related disorder in a subject, the method comprising administering to the subject a therapeutically effective amount of PLCs or derivatives thereof, wherein the PLCs or derivatives thereof express at least one receptor which is a target for a viral protein or a viral particle, which when binds to the receptor is internalized and neutralized to treat or ameliorate the viral disease or a viral disease-related disorder.

31.-42. (canceled)

43. A method for neutralizing a viral infection in a subject comprising:
a. providing PLCs or derivatives thereof comprising an exogenously expressed viral receptor; and
b. administering to the subject a therapeutically effective amount of the PLCs or derivatives thereof in a diluent, a buffer or an excipient.

44. A genetically engineered PLCs or a derivative thereof derived from a PLCs producing progenitor cell where the PLCs are transformed to express at least one receptor, ligand or an antigen which is an entry target of a viral protein or a particle thereof.

45. A PLC or derivative thereof comprising an exogenously expressed viral receptor wherein the receptor is an entry target of a viral protein or a particle thereof, which when binds to the receptor is internalized to neutralize the virus or particles thereof.

46. A diagnostic method or method of screening for an agent, comprising: (a) obtaining a sample from a subject suspected for carrying such agent; (b) admixing with said sample a composition comprising PLCs or derivatives thereof that exogenously or endogenously express one or more receptors, ligands or antigens to which such agents interact with or bind to; (c) and determining the presence or absence of such agents by their binding to the PLCs or derivatives thereof, wherein the agent is selected from one or more of an autoimmune antibody, an or a bacterial or a viral particle, or a viral peptide or a viral nucleic acid in said sample.

47.-50. (canceled)

51. An in-vitro anucleated population of platelet like cells (PLCs) or derivatives thereof possessing one or more of the following characteristics: i) is derived from reprogramming of a somatic cell, progenitor cell or stem cell, the products of which passage through a bioreactor ii) is not a cancerous cell; iii) does not exhibit uncontrolled growth or tumor formation in-vivo; iv) optionally can be systemically administered or has an ability to migrate from a first position to a second position; and (v) has receptors, ligands or antigens, endogenous or exogenous, to bind to autoantibodies, virus, bacteria or toxins for their removal and/or degradation in liver.

52. A method of treating or ameliorating an autoimmune disease, a viral or a bacterial disease or a viral or bacterial disease-related disorder in a subject, the method comprising administering to the subject a therapeutically effective amount of (i) PLCs or derivatives thereof (ii) optionally, extracellular vesicles (iii) in a pharmaceutically acceptable salt or a pharmaceutically acceptable excipient or additive, wherein the PLCs or derivatives thereof or optionally the EVs express at least one receptor, ligand or antigen which is a target for a an autoantibody, a viral or bacterial protein or a viral particle, which when binds to the receptor, ligand or antigen is internalized and neutralized to treat or ameliorate the autoimmune or the viral or bacterial disease or a viral or bacterial disease-related disorder and wherein the PLCs or derivatives thereof are not cancerous cells and/or do not exhibit uncontrolled growth or tumor formation in-vivo.

53. The method of claim 13 wherein the PLCs couple to autoantibodies generated by one or more autoantigens.

54. . The method of claim 53 wherein the autoantigens are selected from one or more of GPIIb/IIIa, α-subunit of Acetylcholine Receptor (AChR), Aquaporin-4, ADAM metallopeptidase with thrombospondin type 1 motif 13 (ADAMTS13), Anti-N-methyl-D-aspartate receptor (anti-NMDAR), Phospholipase A2R and a combination thereof.
